(19) United States
(12) Patent Application Publication
(10) Pub. No.: US 2023/0223150 A1
(43) Pub. Date: Jul. 13, 2023

(54) METHODS AND SYSTEMS FOR PREDICTING ARDS

(71) Applicant: Sime Diagnostics Ltd., London (GB)

(72) Inventors: Peter Schousboe, Odense (DK); Henrik Verder, Bagsværd (DK); Povl Verder, London (GB)

(21) Appl. No.: 17/999,985

(22) PCT Filed: Jun. 4, 2021
(86) PCT No.: PCT/EP2021/065015

§ 371 Date: Nov. 28, 2022

(30) Foreign Application Priority Data
Jun. 4, 2020 (EP) 20178343.8

(51) Int. Cl. G16H 50/30; G01N 33/92; G01N 21/3577; A61K 31/685

(52) U.S. Cl. CPC G16H 50/30; G01N 33/92; G01N 21/3577; A61K 31/685; G01N 2021/3595

(57) ABSTRACT

The present invention relates to methods and systems for predicting the likelihood of acute respiratory distress syndrome (ARDS) in adult subjects. The invention further relates to methods of treatment and identification of subjects with an increased likelihood of developing ARDS as determined by the disclosed methods.

### TECHNICAL FIELD

The present invention relates to methods and systems for predicting the likelihood of acute respiratory distress syndrome (ARDS) in adult subjects. Further disclosed are methods for identification and treatment of subjects with an increased likelihood of developing ARDS as determined by the disclosed methods.

### BACKGROUND

Acute respiratory distress syndrome (ARDS) is a type of respiratory failure characterized by rapid onset of widespread inflammation in the lungs. Symptoms include shortness of breath, rapid breathing, and bluish skin coloration. For those who survive, a decreased quality of life is common.

Causes may include acute inhalation injury, cystic fibrosis, chronic obstructive pulmonary disease, and pneumonia, such as viral or bacterial pneumonia. The underlying mechanism of ARDS involves diffuse injury to cells which form the barrier of the microscopic air sacs of the lungs, surfactant dysfunction, activation of the immune system, and dysfunction of the body's regulation of blood clotting. In effect, ARDS impairs the lungs' ability to exchange oxygen and carbon dioxide.

Globally, ARDS affects approximately 3 million patients annually, accounting for 10% of intensive care unit (ICU) admissions, and 24% of patients receiving mechanical ventilation in the ICU. Despite decades of research, treatment options for ARDS are limited. Supportive care with mechanical ventilation remains the mainstay of management, however therapeutic oxygen and positive-pressure ventilation, while potentially life-saving, can also damage the lung, and the syndrome is still associated with a death rate between 35 and 46%.

There is thus clearly a significant need for earlier and more accurate identification of patients at risk of developing ARDS and improved treatments of such patients. Treatment by administration of surfactant has been suggested, but so far this treatment has been unsuccessful in decreasing the mortality rate, and surfactant administration to treat ARDS is not performed today (Davidson et al., 2006 and Echaide et al., 2017).

Surfactant is a surface-active lipoprotein complex produced by specialized lung cells called Type II cells or Type II pneumocytes. The proteins and lipids that comprise the surfactant have both a hydrophilic region and a hydrophobic region. By adsorbing to the air-water interface of alveoli with the hydrophilic head groups in the water and the hydrophobic tails facing towards the air, the main lipid component of surfactant, dipalmitoylphosphatidylcholine (DPPC), reduces surface tension. In addition to DPPC which constitutes about 40%, the surfactant complex comprises about 40% other phospholipids, about 5% surfactant-associated proteins (SP-A, B, C and D) and additionally cholesterol and trace amounts of other substances.

The function of the surfactant complex is to increase pulmonary compliance, prevent atelectasis (collapse of the lung) at the end of expiration and to facilitate recruitment of collapsed airways.

Surfactant helps prevent collapse of the terminal air-spaces throughout the normal cycle of inhalation and exhalation. The surfactant is packaged by the cell in structures called lamellar bodies, and extruded into the air-spaces. The lamellar bodies subsequently unfold into a complex lining of the air-space. This layer reduces the surface tension of the fluid that lines the air-space. Surface tension is responsible for approximately ⅔ of the elastic recoil forces. In the same way that a bubble will contract to give the smallest surface area for a given volume, so the air/water interface means that the liquid surface will tend towards being as small as possible, thereby causing the air-space to contract. By reducing surface tension, surfactant prevents the air-spaces from completely collapsing on exhalation. In addition, the decreased surface tension allows re-opening of the air-space with a lower amount of force. Therefore, without adequate amounts of surfactant, the air-spaces collapse and are very difficult to expand. Microscopically, a surfactant deficient lung is characterized by collapsed air-spaces alternating with hyper-expanded areas, vascular congestion and, in time, hyaline membranes. Hyaline membranes are composed of fibrin, cellular debris, red blood cells, rare neutrophils and macrophages. They appear as an eosinophilic, amorphous material, lining or filling the air spaces and blocking gas exchange. As a result, blood passing through the lungs is unable to pick up oxygen and unload carbon dioxide. Blood oxygen levels fall and carbon dioxide rises, resulting in rising blood acid levels and hypoxia.

There is currently no specific test to predict whether a subject suffering from a disease that can cause ARDS will progress to develop ARDS.

Methods for diagnosing newborns with respiratory distress syndrome (RDS) by measuring the concentrations and/or activities of lecithin and saturated lecithin, and optionally sphingomyelin, are described in WO 2014/191406, WO 2019/068843 and WO 2019/068848, however this method has not been used for adult diagnosis. Currently, adult diagnosis is based on the physical exam, radiologic imaging of the chest, and blood oxygen levels.

Early treatment of subjects that will go on to develop ARDS, but cannot yet be diagnosed by conventional diagnostic approaches, has the potential to greatly improve the clinical outcome of these patients.

There is thus a need for a rapid method that can be used to predict at an early point in time, whether an adult subject is at risk for developing ARDS, so an appropriate treatment regime can be initiated as soon as possible.

### SUMMARY

The invention is as defined in the claims.

Herein is provided a method for predicting the likelihood of acute respiratory distress syndrome (ARDS) in an adult subject, the method comprising the steps of:
a) providing a sample obtained from said subject,
b) determining in the sample of a), the amount of at least a first and optionally at least a second compound, wherein the first compound is different than the second compound;
c) obtaining a concentration of the first compound and/or a ratio between the first and the second compounds of b); and
d) correlating the concentration of c) with a control concentration, wherein a concentration lower than or equal to the control concentration is indicative of ARDS of the subject; and/or correlating the ratio of c) with a control ratio, wherein a ratio equal to or lower than the control ratio is indicative of ARDS of the subject;

wherein the first compound is lecithin or saturated lecithin, and the second compound is sphingomyelin.

Also provided is a method for predicting the likelihood of acute respiratory distress syndrome (ARDS) in an adult subject, the method comprising the steps of:
a) providing a sample obtained from said subject,
b) determining in the sample of a), the amount of at least a first and at least a second compound, wherein the first compound is different than the second compound;
c) obtaining a ratio between the first and the second compounds of b); and
d) correlating the ratio of c) with a control ratio, wherein a ratio equal to or lower than the control ratio is indicative of ARDS of the subject;

wherein the first compound is lecithin or saturated lecithin, and the second compound is sphingomyelin.

Also provided is a method of predicting the likelihood of an adult subject benefitting from administration of surfactant, the method comprising the steps of:
a) providing a sample obtained from said subject,
b) determining in the sample of a) using suitable analysis means, the amount of at least a first and optionally at least a second compound, wherein the first compound is different than the second compound;
c) obtaining a ratio between the first and the second compounds of b); and
d) correlating the concentration of c) with a control concentration, wherein a concentration lower than or equal to the control concentration is indicative of the subject benefitting from administration of surfactant; and/or correlating the ratio of c) with a control ratio, wherein a ratio equal to or lower than the control ratio is indicative of the subject benefitting from administration of surfactant;

wherein the first compound is lecithin or saturated lecithin, and the second compound is sphingomyelin.

Also provided is a method of monitoring the progression of disease in a subject, said method comprising performing the method as described herein above, wherein said sample is provided from the subject at a set interval, such as every 72 hours or less, such as every 48 hours or less, such as every 24 hours or less, or such as every 12 hours or less.

Also provided is a method for treatment of acute respiratory distress syndrome (ARDS) in an adult subject, the method comprising the steps of:
a) providing a sample obtained from said subject,
b) determining in the sample of a), the amount of at least a first and optionally at least a second compound, wherein the first compound is different than the second compound;
c) obtaining a concentration of the first compound and/or a ratio between the first and the second compounds of b);
d) correlating the concentration of c) with a control concentration, wherein a concentration lower than or equal to the control concentration is indicative of ARDS of the subject; and/or correlating the ratio of c) with a control ratio, wherein a ratio equal to or lower than the control ratio is indicative of ARDS of the subject; and
e) if the concentration of d) is equal to or less than the control value and/or if the ratio of d) is equal to or less than the control ratio, administering a therapeutically effective amount of compound or treatment to said subject.

Also provided is a method for treatment of acute respiratory distress syndrome (ARDS) in an adult subject, the method comprising the steps of:
a) providing a sample obtained from said subject,
b) determining in the sample of a), the amount of at least a first and at least a second compound, wherein the first compound is different than the second compound;
c) obtaining a ratio between the first and the second compounds of b);
d) correlating the ratio of c) with a control ratio, wherein a ratio equal to or lower than the control ratio is indicative of ARDS of the subject; and
e) if the ratio of d) is less than the control ratio, administering a therapeutically effective amount of compound or treatment to said subject.

Also provided is a computer implemented method for predicting the likelihood of ARDS in an adult subject based on spectral data acquired from a sample obtained from said subject, the method comprising the steps of:
a) determining the activity and/or concentration of a first and optionally a second compound in said sample by analyzing said spectral data, wherein the first compound is lecithin or saturated lecithin and the second compound is sphingomyelin;
b) optionally calculating a ratio between the activities and/or concentrations of the first and the second compound; and
c) correlating said concentration with a control value and/or correlating said ratio with a control ratio, wherein a concentration lower than or equal to the control concentration is indicative of ARDS of the subject and/or a ratio equal to or lower than the control ratio is indicative of ARDS of the subject.

Also provided is a computer implemented method for predicting the likelihood of ARDS in an adult subject based on spectral data acquired from a sample obtained from said subject, the method comprising the steps of:
a) determining the activity and/or concentration of a first and a second compounds in said sample by analyzing said spectral data, wherein the first compound is lecithin or saturated lecithin and the second compound is sphingomyelin;
b) calculating a ratio between the activities and/or concentrations of the first and the second compound; and
c) correlating said ratio with a control ratio, wherein a ratio equal to or lower than the control ratio is indicative of ARDS of the subject.

Also provided is a system for predicting the likelihood of ARDS in an adult subject based on a sample obtained from said subject, comprising:
a spectroscope for measuring spectral data from said sample, and
processing means configured for:
a) determining the activity and/or concentration of a first and optionally a second compound in said sample by analyzing said spectral data, wherein the first compound is lecithin or saturated lecithin and the second compound is sphingomyelin;
b) optionally calculating a ratio between the activities and/or concentrations of the first and the second compound;
c) correlating said concentration with a control value and/or correlating said ratio with a control ratio, wherein a concentration lower than or equal to the control concentration is indicative of ARDS of the subject and/or a ratio equal to or lower than the control ratio is indicative of ARDS of the subject; and
d) indicating whether the ratio is equal to or lower than the control ratio, wherein a ratio equal to or lower than a predefined value is indicative of ARDS of the subject.

Also provided is a system for predicting the likelihood of ARDS in an adult subject based on a sample obtained from said subject, comprising:
a spectroscope for measuring spectral data from said sample, and
processing means configured for:
a) determining the activity and/or concentration of a first and a second compounds in said sample by analyzing said spectral data, wherein the first compound is lecithin or saturated lecithin and the second compound is sphingomyelin;
b) calculating a ratio between the activities and/or concentrations of the first and the second compound;
c) correlating said ratio with a control ratio, wherein a ratio equal to or lower than the control ratio is indicative of ARDS of the subject; and
d) indicating whether the ratio is equal to or lower than the control ratio, wherein a ratio equal to or lower than a predefined value is indicative of ARDS of the subject.

Also provided is a computer program product having a computer readable medium, said computer program product being suitable for predicting the likelihood of ARDS in an adult subject based on spectral data acquired from a sample obtained from said subject, said computer program product comprising means for carrying out all the steps of the methods as disclosed herein.

### DESCRIPTION OF DRAWINGS

FIG. 1 shows the dipalmitoylphosphatidylcholine (DPPC) levels (nmol/ml) measured in bronchoalveolar lavage fluid (BALF) in 15 healthy controls (M, 15; mean age, 23; SD, 2 years), in 4 patients with non-COVID-19 ARDS (M/F, 3/1; mean age, 68.5; SD, 3.9 years) and in 11 patients with CARDS (M/F, 10/1; mean age, 65.5; SD, 10.6 years). Boxplots depicts mean values with hinges corresponding to first and third quartile (the 25^{th} and 75^{th} percentiles). Lower and upper whiskers extend to smallest and highest value, respectively. Kruskal-Wallis test: p<0.0001. Abbreviations: CARDS, COVID-19 associated ARDS; DPPC, dipalmitoylphosphatidylcholine; NS, non-significant. ****P<0.0001

FIG. 2 shows a principal component analysis (PCA) score plot with projection of the data onto the span of the principal components (PC). In this analysis, the spectra matrices are decomposed into several PCs by applying singular value decomposition, and a score plot is constructed from the first and second PC. Each PC is a linear combination of the wavenumbers in the spectra. The spectra are baseline corrected (Whittaker smoother) and subsequently normalized by applying standard normal variate (SNV) method to avoid intensity variations in the spectra due to the deposition. Two parameters, lambda (λ=10⁵) and penalty (p=10⁻³), relate to the smoothness of the fit and the penalty imposed to the points giving positive residuals in the fit. The principal components 1 and 2 cover the variances of 80 and 6%, respectively.

### DETAILED DESCRIPTION

### Definitions

The term “surfactant” as used herein refers to pulmonary surfactant as understood by those skilled in the art. Surfactant is a surface-active lipoprotein complex and may function to reduces surface tension of the fluid that lines the air-spaces of the lungs. Surfactant may be produced by specialized lung cells called Type II cells or Type II pneumocytes or it may be synthetically derived.

The term ‘analysis means’ as used herein refers to an instrument capable of detecting the physical property of a molecule or group of molecules. In one embodiment of the disclosure the analysis means is a FTIR spectrometer. Preferably the analysis means is a Bruker alpha FTIR spectrometer capable of performing measurements in very small sample volumes such as down to 1 μL.

The term “Mid-IR” or “Mid wavelength infrared”, also called intermediate infrared (IIR) and mid-red FTIR spectroscopy as used herein refers to light having a wavelength of between about 3 to about 50 μm.

The term “Acute Respiratory Distress Syndrome” as used herein refers to the term as understood by those of skill in the art. ARDS may also be defined as CB00 of WHO's ICD-11 disease classification. The abbreviation ARDS stands for Acute Respiratory Distress Syndrome.

The term “SARS” or “Severe Acute Respiratory Syndrome” as used herein refers to the disease caused by the positive-sense single-stranded RNA virus “severe acute respiratory syndrome-related coronavirus (SARS-CoV)”.

The term “MERS” or “Middle East respiratory syndrome” as used herein to the disease caused by the positive-sense single-stranded RNA virus “Middle East respiratory syndrome related coronavirus (MERS-CoV)”.

The term “COVID-19” or “Coronavirus disease 2019” as used herein refers the disease caused by the positive-sense single-stranded RNA virus “severe acute respiratory syndrome coronavirus 2 (SARS-CoV-2)”.

Method for Predicting the Likelihood of a Subject Developing ARDS

It is important for the success of treatment of acute respiratory distress syndrome (ARDS) in adult subjects to assess the progression and likelihood of developing ARDS as early as possible in the disease stage. Administering treatment as early as possible may greatly improve the clinical outcome of these patients. However, the methods known to date for assessing samples from adult subjects are non-specific and require sample preparation which is time consuming and thus delays commencement of treatment. The methods of the current state of the art furthermore typically rely on samples which can be heterogeneous, thus affecting the reproducibility—and consequently the reliability of the diagnosis.

The present inventors have found that measuring the amount of lecithin or saturated lecithin and optionally of sphingomyelin in the lamellar bodies contained in a sample obtained from an adult subject, can be used to reliably predict the likelihood of said subject developing ARDS. The methods can be performed rapidly and provide reliable and reproducible results.

The present inventors have thus addressed the above problems and found that it is possible to analyze samples from e.g. lung fluid samples obtained from the adult individual and to determine the concentration of lecithin or saturated lecithin and optionally the ratio between lecithin or saturated lecithin and sphingomyelin contained in the samples, without time-consuming laboratory sample preparations.

The inventors have surprisingly found that the ratio between lecithin or saturated lecithin and sphingomyelin as determined by the present methods can reproducibly and reliably be used to predict the likelihood of developing ARDS in adult subjects in such a short time frame that the methods are well suited for point of care units.

In addition, the inventors have found that said ratio may be used to predict the degree of respiratory failure and the level of support that may be needed.

In some aspects is provided a method for predicting the likelihood of acute respiratory distress syndrome (ARDS) in an adult subject, the method comprising the steps of:
a) providing a sample obtained from said subject,
b) determining in the sample of a), the amount of at least a first and optionally at least a second compound, wherein the first compound is different than the second compound;
c) obtaining a concentration of the first compound and/or a ratio between the first and the second compounds of b); and
d) correlating the concentration of c) with a control concentration, wherein a concentration lower than or equal to the control concentration is indicative of ARDS of the subject; and/or correlating the ratio of c) with a control ratio, wherein a ratio equal to or lower than the control ratio is indicative of ARDS of the subject;

wherein the first compound is lecithin or saturated lecithin, and the second compound is sphingomyelin.

In some aspects is provided a method for predicting the likelihood of acute respiratory distress syndrome (ARDS) in an adult subject, the method comprising the steps of:
a) providing a sample obtained from said subject,
b) determining in the sample of a), the amount of at least a first and at least a second compound, wherein the first compound is different than the second compound;
c) obtaining a ratio between the first and the second compounds of b); and
d) correlating the ratio of c) with a control ratio, wherein a ratio equal to or lower than the control ratio is indicative of ARDS of the subject;

wherein the first compound is lecithin or saturated lecithin, and the second compound is sphingomyelin.

In a preferred embodiment, the lecithin is dipalmitoylphosphatidylcholine (DPPC).

In some embodiments, step b) of the present methods comprises the steps of:
i. homogenizing and diluting said sample in a first volume of a first solution, thereby obtaining a homogenous sample;
ii. centrifuging the sample of step i) to obtain a pellet comprising lamellar bodies and a supernatant;
iii. optionally discarding the supernatant and optionally resuspending the pellet in a second volume of a second solution, thereby obtaining a sample for analysis; and
iv. determining the amount of the first compound, and the amount of the second compound, using analysis means.

In some embodiments, step b) of the present methods comprises the steps of:
i. adding an additive such as tris-(2-carboxyethyl)fosfin, (TCEP) to the sample, homogenizing and centrifuging the sample, discarding the supernatant and diluting said sample in a first volume of a first solution, thereby obtaining a homogenous sample;
ii. centrifuging the sample of step i) to obtain a pellet comprising lamellar bodies and a supernatant;
iii. discarding the supernatant and drying the sample; and
iv. determining the amount of the first compound, and the amount of the second compound, using analysis means.

In a clinical setting, the physician utilizing the present methods may, based on the result of the diagnostic method, apply the method of exclusion to determine if the subject wherefrom the sample has been obtained, is suffering or likely to suffer from ARDS. If the result of the method indicates a concentration of the first compound equal to or more than a control value, or if the result of the method indicates a ratio between the first and second compounds significantly above a control ratio, the subject does not suffer from ARDS. If the clinician determines that the condition of the subject is severe, but that the concentration of the first compound or the ratio between the first and second compounds is significantly above a control ratio, the clinician can thus conclude that the subject is likely to suffer from a critical condition other than ARDS, and continue analysis and apply the appropriate treatment.

Sample

The present methods may be performed on a sample selected from a lung epithelial lining fluid sample, a tracheal secretion sample, an oropharyngeal secretion sample, a gastric aspirate sample, a bronchoalveolar lavage sample, a pleural fluid sample, or a blood sample. In some embodiments, the sample is a lung epithelial lining fluid sample. In some embodiments, the sample is a tracheal secretion sample. In some embodiments, the sample is an oropharyngeal secretion sample. In some embodiments, the sample is a gastric aspirate sample. In some embodiments, the sample is a bronchoalveolar lavage sample. In some embodiments, the sample is a pleural fluid sample. Surfactant components can also be detected in the blood. Thus, in some embodiments, the sample is a blood sample.

In some embodiments, the subject has been orally intubated to provide respiratory support. In some embodiments, the sample is obtained less than 96 hours after intubation of the subject, such as less than 72 hours after intubation of the subject, such as less than 48 hours after intubation of the subject, such as less than 24 hours after intubation of the subject, such as less than 12 hours after intubation of the subject. In some embodiments, the sample is obtained between 1 hour and 96 hours after intubation of the subject, such as between 12 hours and 72 hours after intubation of the subject, such as between 24 hours and 72 hours after intubation of the subject. In specific embodiments, the sample is a bronchoalveolar lavage sample.

In some embodiments, the sample provided in step a) of the present methods is homogenous.

In some embodiments, the sample has a volume between 10 and 5000 μL, such as between 20 and 4500 μL, such as between 30 and 4000 μL, such as between 40 and 3500 μL, such as between 50 and 3000 μL, such as between 75 and 2500 μL, such as between 100 and 2000 μL, such as between 125 and 1500 μL, such as between 150 and 1000 μL, such as between 175 and 800 μL, such as between 200 and 600 μL, such as between 225 and 400 μL, such as between 250 and 350 μL, such as about 250 μL, about 300 μL or about 350 μL.

It is preferred that the sample is analyzed directly after sampling to allow for treatment initiation as early as possible. Thus, In some embodiments, the sample is not frozen prior to performing the present methods. The samples may, however, if needed be stored at low temperatures for up to several weeks prior to analysis by the present methods, as storage has been found not to affect the phospholipid content. For example, storage may be for 1 hour or more, such as 2 hours or more, such as 3 hours or more, such as 6 hours or more, such as 12 hours or more, such as 24 hours or more, such as 2 days or more, such as 3 days or more, such as 1 week or more, such as 2 weeks or more, such as 1 month. Storage is preferably at a temperature between 1 and 10° C., such as 2° C., 3° C., 4° C., 5° C., 6° C., 7° C., 8° C., 9° C. or 10° C., preferably at 4° C. or 5° C.

In some embodiments, no sample preparation is required prior to performing the analysis. Thus in some embodiments, the sample is directly transferred from the subject by a sampling means, to the analysis means without intermediate sample preparation.

While it is an advantage for rapid diagnostic purposes to avoid sample preparation, in some embodiments the method further comprises a step of filtering, centrifuging, sonicating, adding an additive such as tris(2-carboxyethyl)phosphine (TCEP) to the sample, and/or diluting the sample prior to loading the sample onto the analysis means, as described herein below.

Sample Preparation

Dilution and Homogenization

In step i) of the current methods, the sample may be diluted and/or homogenized in a first solution. Homogenization can be achieved as is known to the skilled person. For example, homogenization may be performed by pipetting repeatedly or vortexing, thereby strongly stirring the sample. In some embodiments, the sample may be diluted prior to and/or after homogenization.

The sample is diluted in a first volume of a first solution, preferably a hypotonic solution or saline solution. The first solution may be water, such as deionized water, or tap water. In some embodiments of the method, the first solution is deionized water. In some embodiments, the first solution is plain water, such as tap water. In some embodiments, the first solution is saline solution. Without being bound by theory, it is hypothesized that the first solution lyses the cells, thereby facilitating precipitation of lamellar bodies in the next steps of the method.

The volume of the first solution can vary, particularly for embodiments of the present disclosure which comprise precipitation of the sample prior to further analysis steps. The volume of the first solution should preferably be at least equal to half the volume of the sample, such as at least equal to the volume of the sample, such as at least equal to twice the volume of the sample, such as at least equal to three times the volume of the sample, such as at least equal to four times the volume of the sample, such as at least equal to five times the volume of the sample, such as at least equal to 6 times the volume of the sample, such as at least equal to 7 times the volume of the sample, such as at least equal to 8 times the volume of the sample, such as at least equal to 9 times the volume of the sample, such as at least equal to 10 times the volume of the sample, or more. Thus in some embodiments, the ratio of the volume of the sample of step a) to the volume of the first solution used in step i) is about 1:0.5. In another embodiment the ratio of the sample of step i) to the volume of the first solution used in step ii) is about 1:1. In another embodiment the ratio of the sample of step i) to the volume of the first solution used in step ii) is about 1:2. In another embodiment the ratio of the sample of step i) to the volume of the first solution used in step ii) is about 1:3. In another embodiment the ratio of the sample of step i) to the volume of the first solution used in step ii) is about 1:4. In another embodiment the ratio of the sample of step i) to the volume of the first solution used in step ii) is about 1:5. In another embodiment the ratio of the sample of step i) to the volume of the first solution used in step ii) is about 1:6. In another embodiment the ratio of the sample of step i) to the volume of the first solution used in step ii) is about 1:7. In another embodiment the ratio of the sample of step i) to the volume of the first solution used in step ii) is about 1:8. In another embodiment the ratio of the sample of step i) to the volume of the first solution used in step ii) is about 1:9. In another embodiment the ratio of the sample of step i) to the volume of the first solution used in step ii) is about 1:10.

In some embodiments, step i) further comprises centrifuging the sample after homogenization and discarding the supernatant. The centrifugation after homogenization in step i) may be performed at a force between 100 g and 500 g, such as 100 g, 200 g, 300 g, 400 g or 500 g. The centrifugation may be performed for a duration of 1 min to 10 min, such as 2 min to 9 min, such as 3 min to 8 min, such as 3 min to 7 min, such as 3 min, 4 min, 5 min or 6 min. In some embodiments, discarding the supernatant is performed by pipetting the supernatant or by pouring away the supernatant. In some embodiments, the supernatant is instead saved and used a sample for analysis.

In some embodiments, an additive such as tris-(2-carboxyethyl)fosfin, (TCEP) is added to the sample prior to homogenizing in step i).

Precipitation of Lamellar Bodies

In step ii) of the current methods, the homogenous sample obtained in step i) is transferred to a centrifuge, and centrifugation is performed in order to obtain a pellet comprising lamellar bodies and a supernatant. In some embodiments, the supernatant is discarded. In some embodiments, the supernatant is included for subsequent analysis.

In some embodiments, the centrifugation of step ii) is performed at a force between 500 and 10000 g, such as between 1000 and 9000 g, such as between 2000 and 800 g, such as between 3000 and 7000 g, such as between 3500 and 6000 g, such as between 3750 and 5000 g, such as between 3750 and 4500 g, such as at about 4000 g. The centrifugation may be performed for a duration of 1 min to 10 min, such as 2 min to 9 min, such as 3 min to 8 min, such as 4 min to 7 min, such as 4 min, 5 min or 6 min. In some embodiments, the centrifugation of step ii) is performed at 4000 g for 4 min. Following centrifugation, the sample should now present two phases: a solid phase, or pellet, located at the bottom of the tube, and which may be invisible to the naked eye; and a liquid phase, or supernatant. The pellet comprises lamellar bodies from the sample in a concentrated form. The supernatant may also comprise a portion of lamellar bodies; however, the majority of lamellar bodies is preferably present in the pellet.

The supernatant may be discarded. This may be done by pipetting the supernatant away, while being careful not to disturb the pellet, or it may be done by simply gently pouring the supernatant away, and optionally pipetting the remaining volume. The container in which the sample is comprised may be tipped gently and tapped gently on a piece of e.g. absorbing paper, in order to remove the small volumes of liquid which may remain on the walls of the container by gravity. In some embodiments, the supernatant is instead saved and used a sample for analysis.

The pellet is then resuspended in a second volume of a second solution. In some embodiments, resuspending the pellet in step iii) is performed by pipetting repeatedly or vortexing.

The second solution may be a hypotonic solution or a saline solution, so that a sample for analysis is obtained. The second solution may be water, such as deionized water, or tap water. In some embodiments, the second solution is saline solution. In some embodiments, the second solution is deionized water. The second solution may also be plain water, such as tap water.

The volume of second solution to be added to the pellet depends on the analysis means used in step iv). Suitable sample volumes for spectrometer analysis, in particular FTIR analysis, may be between 10 and 200 μL, such as between 25 and 175 μL, such as between 50 and 150 μL, such as between 75 and 125 μL, such as 100 μL, 75 μL, 50 μL, or 25 μL.

Thus, in some embodiments, the second volume is between 10 and 200 μL, such as between 25 and 175 μL, such as between 50 and 150 μL, such as between 75 and 125 μL, such as 100 μL, 75 μL, 50 μL, or 25 μL.

In some embodiments, it may be desirable to remove at least part or all of the second solution after resuspension or prior to analysis. This can be done for example by evaporation of at least part or all of the second solution. In some embodiments, the method thus comprises a step of drying the sample after resuspension and/or prior to determining the amount of the first compound and/or of the second compound.

In some embodiments, the sample for analysis may be transferred to a CaF₂ window. Removal of at least part of the second solution may thus aptly be performed at the time of transfer, for example if the CaF₂ window is at a high temperature allowing for evaporation, such as 80° C. or more, such as 85° C., 86° C., 87° C., 88° C., 89° C., 90° C., 91° C., 92° C., 93° C., 94° C., 95° C. or more. Part of the second solution may also have been evaporated prior to this step, and in some embodiments the method thus comprises two steps of removing at least part of the second solution prior to determining the amounts of the first compound and/or the second compound.

In some embodiments, the method is temperature-independent at least when performed in a temperature range between 20° C. and 40° C.

Sample Analysis

The sample for analysis is used to determine the amount of a first compound selected from lecithin and saturated lecithin, and optionally the amount of a second compound, wherein the second compound is sphingomyelin. In some embodiments, the first compound is dipalmitoylphosphatidylcholine (DPPC), a lecithin.

Without being bound by theory, it is thought that the amounts determined in this step are representative of the amounts present in the original sample, i.e. the sample provided in step i), or are at least proportional to the amounts present in the original sample. Accordingly, the concentration of the first compound in the sample for analysis is preferably equal to or substantially equal to the concentration of the same in the sample provided in step i), i.e. the sample obtained from a subject. The ratio of the first compound to the second compound in the sample for analysis is preferably equal to or substantially equal to the ratio of the same in the sample provided in step i), i.e. the sample obtained from a subject.

In some embodiments, the sample for analysis is analyzed using an infrared spectrometer. In some embodiments, the analysis means is a Fourier transformed infrared spectrometer (FTIR). In some embodiments, the amounts of the first compound and/or the second compound are determined in the mid-wavelength infrared range. In some embodiments, the amounts of the first compound and the second compound are determined in the mid-wavelength infrared range.

In some embodiments, the analysis is performed by Fourier transformed infrared spectroscopy (FTIR) and/or attenuated total reflectance (ATR).

The present methods allow determination of the concentration of the first compound, and optionally of the ratio between the first and second compounds by measuring the amount of the first compound, and optionally of the first and second compounds, respectively. In some embodiments, the present methods allow determination of the ratio between the first and second compounds by measuring the amount of lecithin and sphingomyelin, or by measuring the amount of saturated lecithin and sphingomyelin. The methods may also further include determining the amount of a third compound. In some embodiments, the third compound is apo-hemoglobin. In some embodiments, the third compound is heme. In some embodiments, the third compound is porphyrin. In some preferred embodiments, the third compound is phosphatidylglycerol. In some preferred embodiments, the third compound is hemoglobin.

The amount of a compound can be measured as a concentration or as an activity, as the person of skill is well aware of. Thus, in some embodiments, the amount of the first, second and/or third compound is determined by measuring the activity and/or concentration of the compound(s).

The present methods may be performed rapidly, and are thus well suited for point-of-care units. In some embodiments, the time-to-result is herein defined as the time between step a) and step c) of the present methods. In some embodiments, the time-to-result of the method is between 5 and 60 minutes, such as between 10 and 50 minutes, such as between 20 and 40 minutes, such as between 25 and 35 minutes, such as about 30 minutes. In some embodiments, the time-to-result of the method is 60 minutes or less, such as 55 minutes or less, such as 50 minutes or less, such as 45 minutes or less, such as 40 minutes or less, such as 35 minutes or less, such as 30 minutes or less, such as 25 minutes or less, such as 20 minutes or less, such as 15 minute or less, such as 10 minutes or less, such as 5 minutes or less.

Thus, the methods as described herein allow for initiation of treatment within 5 to 60 minutes, such as within 10 to 50 minutes, such as within 20 to 40 minutes, such as within 25 to 35 minutes, such as within about 30 minutes from providing a sample in step a). In some embodiments, the methods as described herein allow for initiation of treatment within 60 minutes or less, such as within 55 minutes or less, such as within 50 minutes or less, such as within 45 minutes or less, such as within 40 minutes or less, such as within 35 minutes or less, such as within 30 minutes or less, such as within 25 minutes or less, such as within 20 minutes or less, such as within 15 minute or less, such as within 10 minutes or less, such as within 5 minutes or less from providing a sample in step a). In some embodiments, the methods as described herein allow for initiation of treatment within 30 minutes or less from providing a sample in step a). In some embodiments, the methods as described herein allow for initiation of treatment within 20 minutes or less from providing a sample in step a). In some embodiments, the methods as described herein allow for initiation of treatment within 15 minutes or less from providing a sample in step a). In some embodiments, the methods as described herein allow for initiation of treatment within 10 minutes or less from providing a sample in step a).

Prediction of Likelihood of Subject Developing ARDS

Once the amounts of the first compound and/or the second compound have been determined, the concentration of the first compound is calculated and compared with a control value, and/or the ratio between the first compound and the second compound is calculated and compared with a control ratio, as described below. In some embodiments, once the amounts of the first compound and/or the second compound have been determined, the ratio between the first compound and the second compound is calculated and compared with a control ratio, as described below.

In some embodiments, the method further comprises a step of subtracting the amount of the third compound from the amount of the first compound, thus obtaining a background corrected amount of the first compound. In some embodiments, the method further comprises the step of subtracting the amount of the third compound from the amount of the second compound, thus obtaining a background corrected amount of the second compound. In some embodiments, the method further comprises the step of subtracting the amount of the third compound from the amount of both the first and the second compound, thus obtaining a background corrected amount of both the first and the second compound.

The control value is a threshold value, which corresponds substantially to the concentration of the first compound measured in subjects which do not suffer from or are predicted to suffer from ARDS.

In some embodiments, the control value is 50.0 μmol/L±0.5 μmol/L, such as 50.0 μmol/L. A concentration of the first compound equal to or less than 50.0 μmol/L±0.5 μmol/L is indicative of the subject having or being likely to develop ARDS. In some embodiments, the control value is 40.0 μmol/L±0.5 μmol/L, such as 40.0 μmol/L. A concentration of the first compound equal to or less than 40.0 μmol/L±0.5 μmol/L is indicative of the subject having or being likely to develop ARDS. In some embodiments, the control value is 30.0 μmol/L±0.5 μmol/L, such as 30.0 μmol/L. A concentration of the first compound equal to or less than 30.0 μmol/L±0.5 μmol/L is indicative of the subject having or being likely to develop ARDS. In some embodiments, the control value is 25.0 μmol/L±0.5 μmol/L, such as 25.0 μmol/L. A concentration of the first compound equal to or less than 25.0 μmol/L±0.5 μmol/L is indicative of the subject having or being likely to develop ARDS. In some embodiments, the control value is 20.0 μmol/L±0.5 μmol/L, such as 20.0 μmol/L. A concentration of the first compound equal to or less than 20.0 μmol/L±0.5 μmol/L is indicative of the subject having or being likely to develop ARDS. In some embodiments, the control value is 17.5 μmol/L±0.5 μmol/L, such as 17.5 μmol/L. A concentration of the first compound equal to or less than 17.5 μmol/L±0.5 μmol/L is indicative of the subject having or being likely to develop ARDS. In some embodiments, the control value is 15.0 μmol/L±0.5 μmol/L, such as 15.0 μmol/L. A concentration of the first compound equal to or less than 15.0 μmol/L±0.5 μmol/L is indicative of the subject having or being likely to develop ARDS. In some embodiments, the control value is 12.5 μmol/L±0.5 μmol/L, such as 12.5 μmol/L. A concentration of the first compound equal to or less than 12.5 μmol/L±0.5 μmol/L is indicative of the subject having or being likely to develop ARDS. In some embodiments, the control value is 10.0 μmol/L±0.5 μmol/L, such as 10.0 μmol/L. A concentration of the first compound equal to or less than 10.0 μmol/L±0.5 μmol/L is indicative of the subject having or being likely to develop ARDS. In these cases, the subject may be treated accordingly, for example as described herein.

In specific embodiments, the sample is a bronchoalveolar lavage sample and the control value is 25 μmol/L±0.5 μmol/L, such as 25 μmol/L. A concentration of the first compound equal to or less than 25 μmol/L±0.5 μmol/L is indicative of the subject having or being likely to develop ARDS. In specific embodiments, the sample is a bronchoalveolar lavage sample and the control value is 20 μmol/L±0.5 μmol/L, such as 20 μmol/L. A concentration of the first compound equal to or less than 20 μmol/L±0.5 μmol/L is indicative of the subject having or being likely to develop ARDS. In specific embodiments, the sample is a bronchoalveolar lavage sample and the control value is 17.5 μmol/L±0.5 μmol/L, such as 17.5 μmol/L. A concentration of the first compound equal to or less than 17.5 μmol/L±0.5 μmol/L is indicative of the subject having or being likely to develop ARDS. In specific embodiments, the sample is a bronchoalveolar lavage sample and the control value is 15.0 μmol/L±0.5 μmol/L, such as 15.0 μmol/L. A concentration of the first compound equal to or less than 15.0 μmol/L±0.5 μmol/L is indicative of the subject having or being likely to develop ARDS. In specific embodiments, the sample is a bronchoalveolar lavage sample and the control value is 12.5 μmol/L±0.5 μmol/L, such as 12.5 μmol/L. A concentration of the first compound equal to or less than 12.5 μmol/L±0.5 μmol/L is indicative of the subject having or being likely to develop ARDS.

The control ratio is a threshold value, which corresponds substantially to the ratio between the first compound and the second compound measured in subjects which do not suffer from or are predicted to suffer from ARDS.

In some embodiments, the control ratio is between 1.0 and 10.0±0.5, such as between 1.5 and 9.0±0.5, such as between 2.0 and 8.0±0.5, such as between 2.0 and 7.0±0.5, such as between 2.0 and 6.0±0.5, such as 2.5±0.5. In some embodiments, the control ratio is between 2.0 and 5.0±0.5, such as between 2.0 and 4.5±0.5, such as between 2.0 and 4.0±0.5, such as between 2.0 and 3.5±0.5, such as between 2.0 and 3.2±0.5, such as 3.0±0.5.

Accordingly, in some embodiments, if the ratio between the first compound and the second compound is equal to or less than 10.0±0.5 such as equal to or less than 9.5±0.5, such as equal to or less than 9.0±0.5, such as equal to or less than 8.5±0.5, such as equal to or less than 8.0±0.5 the subject is classified as having or likely to develop ARDS. In some embodiments, if the ratio between the first compound and the second compound is equal to or less than 7.5±0.5, such as equal to or less than 7.0±0.5, such as equal to or less than 6.5±0.5, such as equal to or less than 7.0±0.5, such as equal to or less than 6.5±0.5, the subject is classified as having or likely to develop ARDS. In some embodiments, if the ratio between the first compound and the second compound is equal to or less than 6.0±0.5, such as equal to or less than 5.5±0.5, such as equal to or less than 5.0±0.5, such as equal to or less than 4.5±0.5, such as equal to or less than 4.0±0.5, the subject is classified as having or likely to develop ARDS. In some embodiments, if the ratio between the first compound and the second compound is equal to or less than 3.5±0.5, such as equal to or less than 3.0±0.5, such as equal to or less than 2.5±0.5, such as equal to or less than 2.0±0.5, such as equal to or less than 1.5±0.5, such as equal to or less than 1.0±0.5, the subject is classified as having or likely to develop ARDS.

The present methods preferably have a specificity of 50% or more, such as 60% or more, such as 70% or more, such as 80% or more, such as 90% or more. The present methods preferably have a specificity of 75% or more. The present methods preferably have a sensitivity of 50% or more, such as 60% or more, such as 70% or more, such as 80% or more, such as 90% or more. The present methods preferably have a sensitivity of 90% or more.

The present methods may be used to predict the likelihood of a subject developing or suffering from ARDS. In some embodiments, the subject is a human being. In some embodiments, the subject is suffering from a disease or a condition that can cause ARDS.

In some embodiments, said disease or condition is pneumonia, such as viral or bacterial pneumonia. In some embodiments, said disease or condition is acute inhalation injury. Acute inhalation injury may be caused by the inhalation of a toxic compound or gas. In some embodiments, said disease or condition is radiation-induced lung injury. In some embodiments, said disease or condition is aspiration. In some embodiments, said disease or condition is pulmonary embolism. In some embodiments, said disease or condition is atelectasis. In some embodiments, said disease or condition is emphysema. In some embodiments, said disease or condition is cystic fibrosis. In some embodiments, said disease or condition is chronic obstructive pulmonary disease. In some embodiments, said disease or condition is lung contusion. In some embodiments, said disease or condition is chest trauma. In some embodiments, said disease or condition is near-drowning. In some embodiments, said disease or condition is sepsis. In some embodiments, said disease or condition is shock. In some embodiments, said disease or condition is trauma. In some embodiments, said disease or condition is cardiopulmonary bypass. In some embodiments, said disease or condition is transfusion-related acute lung injury. In some embodiments, said disease or condition is burns. In some embodiments, said disease or condition is increased intracranial pressure.

In some embodiments, said disease is caused by an influenza virus. In some embodiments, said disease is caused by a coronavirus, such as a betacoronavirus. In some embodiments, said disease is caused by a betacoronavirus, such as SARS, MERS, or COVID-19. In some embodiments said disease is caused by SARS. In some embodiments said disease is caused by MERS. In some embodiments said disease is caused by COVID-19.

Method of Monitoring the Progression of a Disease in a Subject

In some embodiments, the present invention provides a method of performing serial measurements to predict the likelihood of ARDS in an adult subject over a period of time, said method comprising performing the method as described herein above from step a) to step d), wherein said method from step a) to step d) is repeated for said subject within 72 hours or less, such as within 60 hours or less, or such as within 48 hours or less. In some embodiments, said method from step a) to step d) is repeated within 36 hours or less, such as within 28 hours or less, such as within 20 hours or less, or such as within 16 hours or less. In some embodiments, said method from step a) to step d) is repeated within 24 hours or less. In some embodiments, said method from step a) to step d) is repeated within 12 hours or less. In some embodiments, said method from step a) to step d) is repeated within 18 hours or less, such as within 8 hours or less, or such within 6 hours or less. In some embodiments, said method from step a) to step d) is repeated within 4 hours or less, such as within 3 hours or less, such as within 2 hours or less, such as within 1 hour or less, or such as within 30 minutes or less.

In some embodiments, the present invention provides a method of monitoring the progression of disease in a subject, said method comprising performing the method as described herein above, wherein said sample is provided from the subject at a set interval. Said sample may be provided every 72 hours or less, such as every 60 hours or less, or such as every 48 hours or less. In some embodiments, the sample may be provided every 36 hours or less, such as every 28 hours or less, such as every 20 hours or less, or such as every 16 hours or less. In some embodiments, the sample may be provided every 24 hours or less. In some embodiments, the sample may be provided every 12 hours or less. In some embodiments, the sample may be provided every 18 hours or less, such as every 8 hours or less, or such as every 6 hours or less. In some embodiments, the sample may be provided every 4 hours or less, such as every 3 hours or less, such as every 2 hours or less, such as every 1 hour or less, or such as every 30 minutes or less.

In some embodiments, the methods as disclosed herein are used to evaluate a change in disease status or severity in a subject suffering from a disease that can cause ARDS. In some embodiments, said subject is intubated and the methods as disclosed herein are used to evaluate disease improvement or deterioration. Said evaluation may be used to decide on a relevant treatment regime for the subject. In some embodiments, the methods as disclosed herein may be used to identify or predict a worsening in a disease known to cause ARDS.

In some embodiments, the methods as disclosed herein are used to predict the likelihood of developing ARDS in a subject suffering from disease that can cause ARDS. Thus, the methods as disclosed herein may also be used to identify a subject suffering from a disease that can cause ARDS that is in need of ventilatory support. Accordingly, in some embodiments, the methods as disclosed herein are used to determine the likelihood of a subject requiring ventilatory support.

Method of Predicting the Likelihood of a Subject Benefitting from Administration of Surfactant

It is also an aspect of the present disclosure to provide a method for predicting the likelihood of an adult subject benefitting from administration of surfactant, said method comprising performing the method as disclosed herein,
wherein a concentration determined in step c) lower than or equal to the control concentration is indicative of the subject benefitting from administration of surfactant; and/or wherein a ratio determined in step c) equal to or lower than the control ratio is indicative of the subject benefitting from administration of surfactant.

In some embodiments, the present disclosure thus provides a method of predicting the likelihood of an adult subject benefitting from administration of surfactant, the method comprising the steps of:
a) providing a sample obtained from said subject,
b) determining in the sample of a) using suitable analysis means, the amount of at least a first and optionally at least a second compound, wherein the first compound is different than the second compound;
c) obtaining a concentration of the first compound and/or a ratio between the first and the second compounds of b); and
d) correlating the concentration of c) with a control concentration, wherein a concentration lower than or equal to the control concentration is indicative of the subject benefitting from administration of surfactant; and/or correlating the ratio of c) with a control ratio, wherein a ratio equal to or lower than the control ratio is indicative of the subject benefitting from administration of surfactant;

wherein the first compound is lecithin or saturated lecithin, and the second compound is sphingomyelin.

In some embodiments, the method of predicting the likelihood of an adult subject benefitting from administration of surfactant comprises the steps of:
a) providing a sample obtained from said subject,
b) determining in the sample of a) using suitable analysis means, the amount of at least a first and optionally at least a second compound, wherein the first compound is different than the second compound;
c) obtaining a ratio between the first and the second compounds of b); and
d) correlating the ratio of c) with a control ratio, wherein a ratio equal to or lower than the control ratio is indicative of the subject benefitting from administration of surfactant;

wherein the first compound is lecithin or saturated lecithin, and the second compound is sphingomyelin.

In one embodiment, the present disclosure provides a method for identifying a subject likely to benefit from treatment with surfactant, said method comprising the steps of:
a) providing a sample obtained from said subject,
b) determining in the sample of a) using suitable analysis means, the amount of at least a first and optionally at least a second compound, wherein the first compound is different than the second compound;
c) obtaining a concentration of the first compound and/or a ratio between the first and the second compounds of b); and
d) correlating the concentration of c) with a control concentration, wherein a concentration lower than or equal to the control concentration is indicative of the subject benefitting from administration of surfactant; and/or correlating the ratio of c) with a control ratio, wherein a ratio equal to or lower than the control ratio is indicative of the subject benefitting from administration of surfactant;

wherein the first compound is lecithin or saturated lecithin, and the second compound is sphingomyelin.

In one embodiment, the subject likely to benefit from treatment with surfactant is a subject suffering from non-COVID-19 ARDS.

In one embodiment, the subject likely to benefit from treatment with surfactant is a subject suffering from COVID-19 associated ARDS.

Methods of Treatment of ARDS

Any of the embodiments disclosed herein, i.e. any of the methods described above, may further include a step of treating a subject classified as having an increased likelihood of ARDS and/or an increased likelihood of benefitting from administration of surfactant. In some embodiments, the treatment is administration of a therapeutically effective amount of a medicament to the subject.

Accordingly, also provided herein is a method for treatment of acute respiratory distress syndrome (ARDS) in an adult subject, the method comprising the steps of:
a) providing a sample obtained from said subject,
b) determining in the sample of a), the amount of at least a first and optionally at least a second compound, wherein the first compound is different than the second compound;
c) obtaining a concentration of the first compound and/or a ratio between the first and the second compounds of b);
d) correlating the concentration of c) with a control concentration, wherein a concentration lower than or equal to the control concentration is indicative of ARDS of the subject; and/or correlating the ratio of c) with a control ratio, wherein a ratio equal to or lower than the control ratio is indicative of ARDS of the subject and/or an increased likelihood of the subject benefitting from administration of surfactant; and
e) if the concentration of d) is equal to or less than the control value and/or if the ratio of d) is equal to or less than the control ratio, administering a therapeutically effective amount of compound or treatment to said subject.

Also provided herein is a method for treatment of acute respiratory distress syndrome (ARDS) in an adult subject, the method comprising the steps of:
a) providing a sample obtained from said subject,
b) determining in the sample of a), the amount of at least a first and at least a second compound, wherein the first compound is different than the second compound;
c) obtaining a ratio between the first and the second compounds of b);
d) correlating the ratio of c) with a control ratio, wherein a ratio equal to or lower than the control ratio is indicative of ARDS of the subject and/or an increased likelihood of the subject benefitting from administration of surfactant; and
e) if the ratio of d) is less than the control ratio, administering a therapeutically effective amount of compound or treatment to said subject.

The control value and the control ratio are as defined elsewhere herein.

Based on the diagnostic methods outlined herein above a rapid treatment of the individual in need thereof can be achieved, thus resulting in improved survival rate of the individual.

In some embodiments, the treatment comprises continuous positive airway pressure (CPAP). In some embodiments, the treatment comprises mechanical ventilation, such as airway pressure release ventilation or positive end-expiratory pressure (PEEP) ventilation. In some embodiments, the treatment comprises extracorporeal membrane oxygenation. In some embodiments, the treatment comprises fluid management, such as fluid restriction or inducing diuresis. In some embodiments, the treatment comprises administering a corticosteroid. In some embodiments, the treatment comprises administering nitric oxide. In some embodiments, the treatment comprises administrating supplemental oxygen.

In some embodiments, the treatment comprises administering an antiviral compound, such as remdesivir.

In some preferred embodiments, the treatment comprises administering surfactant.

In some embodiments, the treatment comprises administering one or more components of surfactant, such as dipalmitoylphosphatidylcholine (DPPC), phosphatidylcholine, phosphatidylglycerol, cholesterol, surfactant protein A, surfactant protein B, surfactant protein C and/or surfactant protein D. Thus, in some embodiments, the treatment comprises administering dipalmitoylphosphatidylcholine (DPPC). In some embodiments, the treatment comprises administering phosphatidylcholine. In some embodiments, the treatment comprises administering phosphatidylglycerol. In some embodiments, the treatment comprises administering cholesterol. In some embodiments, the treatment comprises administering surfactant protein A. In some embodiments, the treatment comprises administering surfactant protein B. In some embodiments, the treatment comprises administering surfactant protein C. In some embodiments, the treatment comprises administering surfactant protein D.

In some embodiments, said surfactant is a synthetic surfactant. In some embodiments, the surfactant is an animal-derived surfactant. In some embodiments, the surfactant is a combination of a synthetic surfactant and an animal-derived surfactant.

Surfactant or one or more components thereof may be administered by nebulization or bolus.

Thus, provided in the present disclosure, is surfactant for use in the treatment of ARDS in a subject, wherein the surfactant is administered to a subject having an increased likelihood of ARDS as determined by any of the methods as disclosed herein.

Also provided in the present disclosure is use of a surfactant for the manufacture of a medicament for the treatment of ARDS in a subject, wherein the surfactant is administered to a subject having an increased likelihood of ARDS as determined by any of the methods as disclosed herein.

Computer Implemented Methods and Systems for Diagnosing RDS

In some aspects, the invention discloses a computer implemented method for predicting the likelihood of ARDS in an adult subject based on spectral data acquired from a sample obtained from said subject, the method comprising the steps of:
a) determining the activity and/or concentration of a first and optionally a second compound in said sample by analyzing said spectral data, wherein the first compound is lecithin or saturated lecithin and the second compound is sphingomyelin;
b) optionally calculating a ratio between the activities and/or concentrations of the first and the second compound; and
c) correlating said concentration with a control value and/or correlating said ratio with a control ratio, wherein a concentration lower than or equal to the control concentration is indicative of ARDS of the subject and/or a ratio equal to or lower than the control ratio is indicative of ARDS of the subject.

Also disclosed is a computer implemented method of predicting the likelihood of an adult subject benefitting from administration of surfactant, based on spectral data acquired from a sample obtained from said subject, the method comprising the steps of:
a) determining the activity and/or concentration of a first and optionally a second compound in said sample by analyzing said spectral data, wherein the first compound is lecithin or saturated lecithin and the second compound is sphingomyelin;
b) optionally calculating a ratio between the activities and/or concentrations of the first and the second compound; and
c) correlating said concentration with a control value and/or correlating said ratio with a control ratio, wherein a concentration lower than or equal to the control concentration is indicative of the subject benefitting from administration of surfactant and/or a ratio equal to or lower than the control ratio is indicative of the subject benefitting from administration of surfactant.

In some aspects, the invention provides a computer implemented method for predicting the likelihood of ARDS in an adult subject based on spectral data acquired from a sample obtained from said subject, the method comprising the steps of:
a) determining the activity and/or concentration of a first and a second compounds in said sample by analyzing said spectral data, wherein the first compound is lecithin or saturated lecithin and the second compound is sphingomyelin;
b) calculating a ratio between the activities and/or concentrations of the first and the second compound; and
c) correlating said ratio with a control ratio, wherein a ratio equal to or lower than the control ratio is indicative of ARDS of the subject.

In some embodiments, the invention provides a computer implemented method for predicting the likelihood of an adult subject benefitting from administration of surfactant, based on spectral data acquired from a sample obtained from said subject, the method comprising the steps of:
a) determining the activity and/or concentration of a first and a second compounds in said sample by analyzing said spectral data, wherein the first compound is lecithin or saturated lecithin and the second compound is sphingomyelin;
b) calculating a ratio between the activities and/or concentrations of the first and the second compound; and
c) correlating said ratio with a control ratio, wherein a ratio equal to or lower than the control ratio is indicative of the subject benefitting from administration of surfactant.

Steps a), b) and c), may be performed by any of the methods as described herein above.

The control value and the control ratio are as defined elsewhere herein.

As time may be an important factor for treating ARDS, the computer-implemented prediction method may advantageously be integrated in a prediction system that can be installed in hospital departments. Such a system can integrate spectroscopy, analysis and disease indication that may provide a prediction within minutes after a biological sample has been obtained.

Accordingly, provided herein is a system for predicting the likelihood of ARDS in an adult subject based on a sample obtained from said subject, comprising:
a spectroscope for measuring spectral data from said sample, and
processing means configured for:
a) determining the activity and/or concentration of a first and optionally a second compound in said sample by analyzing said spectral data, wherein the first compound is lecithin or saturated lecithin and the second compound is sphingomyelin;
b) optionally calculating a ratio between the activities and/or concentrations of the first and the second compound;
c) correlating said concentration with a control value and/or correlating said ratio with a control ratio, wherein a concentration lower than or equal to the control concentration is indicative of ARDS of the subject and/or a ratio equal to or lower than the control ratio is indicative of ARDS of the subject; and
d) indicating whether the ratio is equal to or lower than the control ratio, wherein a ratio equal to or lower than a predefined value is indicative of ARDS of the subject.

Also provided herein is a system for predicting the likelihood of an adult subject benefiting from administration of surfactant, based on a sample obtained from said subject, comprising:
a spectroscope for measuring spectral data from said sample, and
processing means configured for:
a) determining the activity and/or concentration of a first and optionally a second compound in said sample by analyzing said spectral data, wherein the first compound is lecithin or saturated lecithin and the second compound is sphingomyelin;
b) optionally calculating a ratio between the activities and/or concentrations of the first and the second compound;
c) correlating said concentration with a control value and/or correlating said ratio with a control ratio, wherein a concentration lower than or equal to the control concentration is indicative of the subject benefitting from administration of surfactant and/or a ratio equal to or lower than the control ratio is indicative of the subject benefitting from administration of surfactant; and
d) indicating whether the ratio is equal to or lower than the control ratio, wherein a ratio equal to or lower than a predefined value is indicative of the subject benefitting from administration of surfactant.

Also provided herein is a system for predicting the likelihood of ARDS in an adult subject based on a sample obtained from said subject, comprising:
a spectroscope for measuring spectral data from said sample, and
processing means configured for:
a) determining the activity and/or concentration of a first and a second compounds in said sample by analyzing said spectral data, wherein the first compound is lecithin or saturated lecithin and the second compound is sphingomyelin;
b) calculating a ratio between the activities and/or concentrations of the first and the second compound;
c) correlating said ratio with a control ratio, wherein a ratio equal to or lower than the control ratio is indicative of ARDS of the subject; and
d) indicating whether the ratio is equal to or lower than the control ratio, wherein a ratio equal to or lower than a predefined value is indicative of ARDS of the subject.

Additionally provided herein is a system for predicting the likelihood of an adult subject benefiting from administration of surfactant based on a sample obtained from said subject, comprising:
a spectroscope for measuring spectral data from said sample, and
processing means configured for:
a) determining the activity and/or concentration of a first and a second compounds in said sample by analyzing said spectral data, wherein the first compound is lecithin or saturated lecithin and the second compound is sphingomyelin;
b) calculating a ratio between the activities and/or concentrations of the first and the second compound;
c) correlating said ratio with a control ratio, wherein a ratio equal to or lower than the control ratio is indicative of the subject benefitting from administration of surfactant; and
d) indicating whether the ratio is equal to or lower than the control ratio, wherein a ratio equal to or lower than a predefined value is indicative of the subject benefitting from administration of surfactant.

Thus, the present methods may be integrated in a personal computer or they may be effectuated from a website, mobile phone, smartphone or other electronic device capable of executing computer code. A further embodiment of the invention therefore relates to a computer program product having a computer readable medium, said computer program product being suitable for predicting the likelihood of ARDS in an adult subject based on spectral data acquired from a sample obtained from said subject, said computer program product comprising means for carrying out all the steps of the methods as disclosed herein above.

The system may be part of a health monitoring system as described in WO 2008/019695 disclosing a health monitoring service based on a central server, wherein the measurement of the samples is carried out as a local measurement and the measurement data are subsequently sent to a central server, where the data are processed and analyzed, for example by expert knowledge systems, and a health profile is generated and sent back to the local system. Thus, the processing means may be fully or partly integrated in a central service remote from the local hospital department or even remote from the hospital. However, the processing means may also be fully integrated in the local system such that the system located in the hospital department includes spectrometer, spectral analysis and processing and disease indication.

Disclosed herein is thus also the use of a system in a method for predicting the likelihood of ARDS in an adult subject according to any of the methods disclosed herein, said system comprising:
a spectroscope for measuring spectral data from said sample, and
processing means configured for:
a) determining the activity and/or concentration of a first and optionally a second compound in said sample by analyzing said spectral data, wherein the first compound is lecithin or saturated lecithin and the second compound is sphingomyelin;
b) optionally calculating a ratio between the activities and/or concentrations of the first and the second compound;
c) correlating said concentration with a control value and/or correlating said ratio with a control ratio, wherein a concentration lower than or equal to the control concentration is indicative of ARDS of the subject and/or a ratio equal to or lower than the control ratio is indicative of ARDS of the subject; and
d) indicating whether the ratio is equal to or lower than the control ratio, wherein a ratio equal to or lower than a predefined value is indicative of ARDS of the subject.

In some embodiments is disclosed the use of a system in a method for predicting the likelihood of an adult subject benefitting from administration of surfactant according to any of the methods disclosed herein, said system comprising:
a spectroscope for measuring spectral data from said sample, and
processing means configured for:
a) determining the activity and/or concentration of a first and optionally a second compound in said sample by analyzing said spectral data, wherein the first compound is lecithin or saturated lecithin and the second compound is sphingomyelin;
b) optionally calculating a ratio between the activities and/or concentrations of the first and the second compound;
c) correlating said concentration with a control value and/or correlating said ratio with a control ratio, wherein a concentration lower than or equal to the control concentration is indicative of the subject benefitting from administration of surfactant and/or a ratio equal to or lower than the control ratio is indicative of the subject benefitting from administration of surfactant; and
d) indicating whether the ratio is equal to or lower than the control ratio, wherein a ratio equal to or lower than a predefined value is indicative of the subject benefitting from administration of surfactant.

Also disclosed herein is the use of a system in a method for predicting the likelihood of ARDS in an adult subject according to any of the methods disclosed herein, said system comprising:
a spectroscope for measuring spectral data from said sample, and
processing means configured for:
a) determining the activity and/or concentration of a first and a second compounds in said sample by analyzing said spectral data, wherein the first compound is different than the second compound and the first compound is lecithin or saturated lecithin and the second compound is sphingomyelin;
b) calculating a ratio between the activities and/or concentrations of the first and the second compounds;
c) correlating said ratio with a control ratio; and
d) indicating whether the ratio is equal to or lower than the control ratio, wherein a ratio equal to or lower than a predefined value is indicative of ARDS of the subject.

Also disclosed herein is the use of a system in a method for predicting the likelihood of an adult subject benefitting from administration of surfactant according to any of the methods disclosed herein, said system comprising:
a spectroscope for measuring spectral data from said sample, and
processing means configured for:
a) determining the activity and/or concentration of a first and a second compounds in said sample by analyzing said spectral data, wherein the first compound is different than the second compound and the first compound is lecithin or saturated lecithin and the second compound is sphingomyelin;
b) calculating a ratio between the activities and/or concentrations of the first and the second compounds;
c) correlating said ratio with a control ratio; and
d) indicating whether the ratio is equal to or lower than the control ratio, wherein a ratio equal to or lower than a predefined value is indicative of the subject benefitting from administration of surfactant.

### REFERENCES

Davidson, W. J., Dorscheid, D., Spragg, R., Schulzer, M., Mak, E. and Ayas, N. T., 2006. Exogenous pulmonary surfactant for the treatment of adult patients with acute respiratory distress syndrome: results of a meta-analysis. Critical Care, 10(2), p. R41.
Echaide, M., Autilio, C., Arroyo, R. and Perez-Gil, J., 2017. Restoring pulmonary surfactant membranes and films at the respiratory surface. Biochimica et Biophysica Acta (BBA)-Biomembranes, 1859(9), pp. 1725-1739.
Heiring C, Verder H, Schousboe P, Jessen T E, Bender L, Ebbesen F, Dahl M, Eschen C, Fenger-Gron J, Hoskuldsson A, Matthews M, Reinholdt J, Scoutaris N, Smedegaard H. Predicting respiratory distress syndrome at birth using a fast test based on spectroscopy of gastric aspirates: 2. Clinical part. Acta Paediatr 2020; 109: 285-290.
Lan J, Ge J, Yu J, Shan S, Zhou H, Fan S, Zhang Q, Shi X, Wang Q, Zhang L, Wang X. Structure of the SARS-CoV-2 spike receptor-binding domain bound to the ACE2 receptor. Nature 2020; 581: 215-220.
Plovsing R R, Berg R M, Evans K A, Konge L, Iversen M, Garred P, Moller K. Transcompartmental inflammatory responses in humans: IV versus endobronchial administration of endotoxin*. Crit Care Med 2014; 42: 1658-1665.
Ronit A, Berg R M G, Bay J T, Haugaard A K, Ahlstrom M G, Burgdorf K S, Ullum H, Rorvig S B, Tjelle K, Foss N B, Benfield T, Marquart H V, Plovsing R R. Compartmental immunophenotyping in COVID-19 ARDS: A case series. J Allergy Clin Immunol 2021; 147: 81-91.
Rugonyi S, Biswas S C, Hall S B. The biophysical function of pulmonary surfactant. Respir Physiol Neurobiol 2008; 163: 244-255.
Taut F J H, Rippin G, Schenk P, Findlay G, Wurst W, Hafner D, Lewis J F, Seeger W, Gunther A, Spragg R G. A Search for subgroups of patients with ARDS who may benefit from surfactant replacement therapy: a pooled analysis of five studies with recombinant surfactant protein-C surfactant (Venticute). Chest 2008; 134: 724-732.
Xu Z, Shi L, Wang Y, Zhang J, Huang L, Zhang C, Liu S, Zhao P, Liu H, Zhu L, Tai Y, Bai C, Gao T, Song J, Xia P, Dong J, Zhao J, Wang F S. Pathological findings of COVID-19 associated with acute respiratory distress syndrome. Lancet Respir Med 2020; 8: 420-422.

### Items

1. A method for predicting the likelihood of acute respiratory distress syndrome (ARDS) in an adult subject, the method comprising the steps of:
   a) providing a sample obtained from said subject,
   b) determining in the sample of a) using suitable analysis means, the amount of at least a first and optionally at least a second compound, wherein the first compound is different than the second compound;
   c) obtaining a concentration of the first compound and/or a ratio between the first and the second compounds of b); and
   d) correlating the concentration of c) with a control concentration, wherein a concentration lower than or equal to the control concentration is indicative of ARDS of the subject; and/or correlating the ratio of c) with a control ratio, wherein a ratio equal to or lower than the control ratio is indicative of ARDS of the subject;
wherein the first compound is lecithin or saturated lecithin, and the second compound is sphingomyelin.
2. The method according to any one of the preceding items, wherein the subject is suffering from a disease or a condition that can cause ARDS.
3. The method according to item 2, wherein said disease or condition is selected from the group consisting of pneumonia, such as viral or bacterial pneumonia, acute inhalation injury, radiation-induced lung injury, aspiration, pulmonary embolism, atelectasis, emphysema, cystic fibrosis, chronic obstructive pulmonary disease, lung contusion, chest trauma, near-drowning, sepsis, shock, trauma, cardiopulmonary bypass, transfusion-related acute lung injury, burns, and increased intracranial pressure.
4. The method according to any one of the preceding items, wherein the sample is a lung epithelial lining fluid sample, a tracheal secretion sample, an oropharyngeal secretion sample, a gastric aspirate sample, a bronchoalveolar lavage sample, a pleural fluid sample, or a blood sample.
5. The method according to any one of the preceding items, wherein step b) comprises the steps of:
   i. homogenising and diluting said sample in a first volume of a first solution, thereby obtaining a homogenous sample;
   ii. centrifuging the sample of step i) to obtain a pellet comprising lamellar bodies and a supernatant;
   iii. optionally discarding the supernatant and optionally resuspending the pellet in a second volume of a second solution, thereby obtaining a sample for analysis; and
   iv. determining the amount of the first compound, and the amount of the second compound, using analysis means.
6. The method according to any one of the preceding items, wherein the analysis means is an infrared spectrometer, such as a Fourier transformed infrared (FTIR) spectrometer.
7. The method according to any one of the preceding items, wherein the time-to-result of the method is less than 10 minutes, such as about 5 minutes.
8. The method according to any one of the preceding items, wherein the method allows for initiation of treatment within 60 minutes or less, such as within 55 minutes or less, such as within 50 minutes or less, such as within 45 minutes or less, such as within 40 minutes or less, such as within 35 minutes or less, such as within 30 minutes or less, such as within 25 minutes or less, such as within 20 minutes or less, such as within 15 minute or less, such as within 10 minutes or less, such as within 5 minutes or less from providing a sample in step a).
9. The method according to any one of the preceding items, wherein if the ratio between the first compound and the second compound is equal to or less than 10.0±0.5, such as equal to or less than 9.0±0.5, such as equal to or less than 8.0±0.5, such as equal to or less than 7.0±0.5, such as equal to or less than 6.0±0.5, such as equal to or less than 5.0±0.5, such as equal to or less than 4.0±0.5, such as equal to or less than 3.0±0.5, such as equal to or less than 2.0±0.5, or such as equal to or less than 1.0±0.5, the subject is classified as having or likely to develop ARDS.
10. The method according to any one of the preceding items, wherein if the concentration of the first compound is equal to or less than 50 μM±0.5 μM, equal to or less than 40 μM±0.5 μM, equal to or less than 30±0.5 μM, equal to or less than 20±0.5 μM, or equal to or less than 10±0.5 μM, the subject is classified as having or likely to develop ARDS.
11. A method of monitoring the progression of disease in a subject, said method comprising performing the method according to any one of the preceding items, wherein said sample is provided from the subject every 72 hours or less, such as every 60 hours or less, such as every 48 hours or less, such as every 36 hours or less, such as every 24 hours or less, such as every 20 hours or less, such as every 16 hours or less, such as every 12 hours or less, such as every 8 hours or less, such as every 6 hours or less, such as every 4 hours or less, such as every 2 hours or less, such as every 1 hour or less.
12. The method according to any one of the preceding items, wherein said method is used to determine the likelihood of said subject requiring ventilatory support.
13. A computer implemented method for predicting the likelihood of ARDS in an adult subject based on spectral data acquired from a sample obtained from said subject, the method comprising the steps of:
   a) determining the activity and/or concentration of a first and optionally a second compound in said sample by analysing said spectral data, wherein the first compound is lecithin or saturated lecithin and the second compound is sphingomyelin;
   b) optionally calculating a ratio between the activities and/or concentrations of the first and the second compound;
   c) correlating said concentration with a control value and/or correlating said ratio with a control ratio, wherein a concentration lower than or equal to the control concentration is indicative of ARDS of the subject and/or a ratio equal to or lower than the control ratio is indicative of ARDS of the subject.
14. The method according to item 13, further comprising the features of any of items 1 to 10.
15. Surfactant for use in the treatment of ARDS in a subject, wherein the surfactant is administered to a subject having an increased likelihood of ARDS as determined by any of items 1 to 10.

### EXAMPLES

### Example 1—Surfactant in COVID19 Patients Requiring Ventilatory Support

The present study is being conducted to investigate whether surfactant is measurable in tracheal secretions from COVID-19 patients and, furthermore, if it is reduced in those patients requiring ventilator support as compared to non-COVID19 patients. The study also aims to clarify if dysfunctional surfactant in COVID19 patients regains its function when respiratory failure improves, and if surfactant levels can thus be used as a readout for assessing and predicating the likelihood of ARDS.

COVID19 can cause ARDS. It is thus possible to predict the degree of respiratory failure and the level of support that may be needed based on the level of surfactant.

Subject Eligibility

Population

The main population is COVID19 patients. Surfactant levels measured in COVID-19 patients will be compared to a matched cohort of intubated non-COVID-19 patients.

Inclusion Criteria
Patients positive for coronavirus 2 (SARS-CoV-2) OR non-COVID19 patient AND
Respiratory failure requiring mechanical ventilation support OR respiratory failure with need for non-invasive ventilatory support AND
Age >18 years

The non-COVID19 (n=10) will serve as a comparable matched cohort of intubated COVID-19 patients (n=20).

Exclusion Criteria

Inability to obtain tracheal secretions (TS) as determined by clinical judgement by responsible clinician.

Patients with known surfactant dysfunction (such as mutations in the SFTPB or ABCA3 gene).

Outcome Measures

Primary outcome is the level of surfactant in lung fluid.

Sample Size

It is estimated that at least 20 patients will be needed to be included to be able to determine various levels of surfactant in tracheal secretions (TS) and/or in bronchoalveolar lavage (BL) fluid.

Study Design

Single-center, exploratory, non-interventional exploratory pilot-like study design.

The aim is to include twenty patients for consecutive sampling of lung fluid secretes by TS and BL when considered clinical feasible and relevant. Using a prospective design patients will be included from local hospitals in Denmark.

Tracheal Secretions and Bronchiolar Lavage Methodology

When it is applicable in relation to patient stability and in accordance with the clinical condition of the patient and in agreement with the treating clinician, TS will be obtained on a twice daily schedule. In some patients bacterial superinfection may be suspected by the clinician in charge of treatment. In such patients a BL may be considered to be clinical relevant as lung fluid needs to be cultured for potential bacterial growth. When it is applicable in daily routine and only when BL is considered clinically relevant, lung fluid obtained by BL will also be included in the current study.

Methods to Estimate Pulmonary Surfactant Status by FTIR

Lung Fluid Preparation

Fresh sample-material (BL, TS or similar) is liquidized by shaking in saline or pure water. Briefly, the method contains the steps:

Sample collection, sample size depends on volume
1. Pre-treatment with tris-(2-carboxyethyl)fosfin, (TCEP) (optional)
2. Shake sample
3. Pre-centrifugation 300×g, 3 minutes (optional)
4. decanting supernatant
5. Pipetting 0.1 ml
6. Dilution with 0.5 ml Water (mixing)
7. Centrifugation 4000×g for 4 minutes
8. Collecting precipitate (discarding supernatant)
9. Drying sample for 1-2 minutes
10. Apply to FTIR, dry transmission
11. Read result in real time
12. AI algorithm result with ratio between lecithin or saturated lecithin and sphingomyelin (L/S ratio)

Under conditions where the viscosity is high, TCEP can be added to the samples. TCEP does not interfere with the spectroscopic data or the surfactant, but splits proteinaceous accumulations that may be difficult to dissolve during the dilution processes. The method may also require a pre-centrifugation step to remove cells/cellular material at 300 g for 3 minutes and discarding of precipitate which has negligible influence on the surfactant concentration. The samples are currently handled and analyzed by an automated Alpha Plus Data Engine from SIME Diagnostics Limited.

Data Generation and Analysis Plan

Data generation and analysis will be performed as outlined in Table 1, below.

TABLE 1
Data generation and analysis plan.

| | | DATA | | | |
|---|---|---|---|---|---|
| | INPUT | GENERATION | ANALYSIS | OUTPUT | HYPOTHESIS |
| 1a | BL | Mid-infrared FTIR | Machine | L/S Ratio | Establish if existing |
| | | spectroscopy, dry | learning | and DPPC | algorithms can measure |
| | | transmission | | | surfactant levels in BL |
| 1b | Blood | Mid-infrared FTIR | Machine | Spectral | Spectral analysis of blood |
| | | spectroscopy, dry | learning | correlations | plasma combined with |
| | | transmission | | | patient data may provide |
| | | | | | us with the ability to spot |
| | | | | | patients with ventilation |
| | | | | | issues. |
| 2 | BL & | Mid-infrared FTIR | Artificial | Spectral | Combining 1b with spectra |
| | Blood | spectroscopy, dry | intelligence | correlations | of BL may provide also be |
| | | transmission | | | a possibility to find |
| | | | | | correlations that may |
| | | | | | distinguish between patients |
| | | | | | suitable for ventilation. |
| 3 | BL & | Mid-infrared FTIR | ML/AI | Spectral | Establish if spectral and |
| | Blood + | spectroscopy, dry | | correlations | patient data combined |
| | Patient | transmission | | combined | enables the generation of |
| | | PLUS | | with patient | an algorithm that can |
| | | Patient, treatment | | data | distinguish patients suitable |
| | | and outcomes data | | | for ventilation combined |
| | | | | | with blood and/or surfactant |
| | | | | | spectra. |
| 4 | BL | Elisa | Chemical | Surfactant | Establish SP-A, B, C and |
| | | | reagents | protein-D | D measurements on BL to |
| | | | | (SP-D) | elucidate the levels as |
| | | | | | response to inflammation |
| | | | | | in Covid19 affected lungs. |
| | | | | | Furthermore, SP-B and -A |
| | | | | | levels can be compared with |
| | | | | | previous data, published |
| | | | | | by others. |
| | | | | | Phospholipase A2 will be |
| | | | | | included in the analysis |
| | | | | | to investigate the turnover |
| | | | | | of phospholipids. |
| 5 | BL/TS | RT-PCR | miRNA | Signalling | Measure microRNA to |
| | | | | | estimate a potential |
| | | | | | regulatory mechanism in |
| | | | | | the expression of surfactant |

Data Processing and Analysis of Surfactant

A data-driven approach will be employed to process and analyze data generated from the study. Primary data consisting of biochemical FTIR spectra from BL and blood will be combined with secondary data consisting of specific clinical data points. The combination of these primary and secondary data creates unique highly multivariate datasets which will be analyzed by AI and compared with clinical treatment and outcomes data. Spectral data from BL will also analyzed by multiple pre-existing machine learning classifiers and algorithms to determine surfactant concentrations which may also be included in the multivariate datasets for AI analysis. These data combinations may potentially enhance the overall picture of patient status (improvement and/or deterioration) and unveil data with significant impact on patient condition. Data analysis tools that will be employed include:
Partial Least Square: Method for multivariate analysis where data is uncertain
Support Vector Machine: Model and algorithms for two-group classification problems
Probabilistic AI: Framework used to model predictions of future data
Surfactant algorithms: FTIR classifiers for dipalmitoylphosphatidylcholine, sphingomyelin
R studio: Software modelling and analysis platform

Blood Sampling

When lung fluid is sampled the aim is to obtain venous blood (5 ml) from a catheter which has been inserted into the patient. Blood will be allowed to coagulate and serum separated. The samples will then be centrifuged and serum separated into small tubes and stored in a freezer at −20° C. to be transferred to −80° C. Serum will used for analysis of surfactant protein fragments using commercial available sandwich and ELISA technique (Winkler).

Clinical Data

Data is continuously recorded in the case report form (CRF) and entered into the study database to which only the investigators has the password. Data is not directly personally identifiable and protected under the Law on Processing of Personal Data and the Health Law. The code for the identification of patients is saved in a word document. All documents will be stored for 10 years. The study will be reported to the Data Protection Agency.

The following data are planned to be sampled:
Respiratory variables such as TV, PEEP, FiO₂, PaO₂.
Respiratory failure in accordance to ARDS severity classification
Gender, age, weight, height
Underlying health conditions
Treatment specific data (such as use of anti-viral treatment)
Other relevant data, blood sample data such as parameters of infection in blood
Lung fluid and serum

The level of respiratory system compliance will be recorded as well as whether patients are considered recruitable. Use of ventilatory support device is recorded (CPAP, NIV, mechanical ventilation) in addition to the use of prone position for ventilation. Permission from relevant authorities to obtain data from the Danish healthcare platform will be obtained.

Statistical Analysis

Patients will act as their own control in a before-after like study design although group comparisons are being planned. A suggestion is that an evaluation of surfactant is done with patients grouped in accordance to their ARDS severity (mild ARDS 200<PaO₂/FiO₂≤300 mmHg, moderate ARDS 100<PaO₂/FiO₂≤200 mmHg, severe ARDS PaO₂/FiO₂≤100 mmHg; ref. 30), ICU length or stay as well as max PEEP levels applied for an unspecified not predefined number of days. Clinical factors correlated to COVID-19 patients will be determined by t-test for continuous variables and chi-square test for categorical variables. Paired Cox-Wilcoxon test will be used for spectra analyses. Two-tailed p-values <0.05 will be considered to indicate statistical significance.

### Example 2—Reduced Levels of Pulmonary Surfactant in COVID-19 ARDS

Introduction

Like other coronaviruses, severe acute respiratory syndrome coronavirus 2 (SARS-CoV-2) uses the angiotensin-converting enzyme-2 (ACE2) receptor to access and infect pulmonary surfactant producing alveolar type II (ATII) cells (Lan et al., 2020). Importantly, the lungs of patients with coronavirus disease 2019 (COVID-19)-associated acute respiratory distress syndrome (CARDS) exhibit diffuse alveolar damage, protein leak, and hyaline membrane formation (Ronit et al., 2021 and Xu et al., 2020), which may involve loss of surfactant caused by virus-induced lysis or apoptosis of ATII cells.

Pulmonary surfactant consists mainly of dipalmitoylphosphatidylcholine (DPPC) that functions to reduce surface tension, thus stabilizing the alveoli, while increasing pulmonary compliance and reducing the work of breathing (Rugonyi et al., 2008). Although exogenous surfactant therapy is effective for premature new-borns with respiratory distress syndrome (RDS), it has failed to improve mortality in non-COVID-19 ARDS (Taut et al., 2008). However, CARDS may be associated with an earlier and more profound loss of surfactant, and clinical trials are currently underway to evaluate the effectiveness of exogenous surfactant.

Here, we assessed DPPC levels in bronchoalveolar lavage fluid (BALF) in COVID-19 patients with moderate-to-severe ARDS.

Materials and Methods

The study was approved by relevant authorities and registered at ClinicalTrials.gov (NCT04354584). A total of 11 CARDS patients were included in the present study. Data on compartmental immunophenotyping on 4 of the patients have previously been reported elsewhere (Ronit et al., 2021). Furthermore, 4 non-COVID-19 patients with moderate-to-severe ARDS and 15 healthy controls from a previous study (Plovsing et al., 2014) were included for comparison. Since all patients were incompetent, informed consent was obtained from next of kin, and the BAL procedure was performed within less than 72 hours of mechanical ventilation.

The BAL procedure was performed as previously described (Ronit et al., 2020 and Plovsing et al., 2014). Three successive 50-ml aliquots of isotonic saline were instilled in the medial segment of the right middle lobe and aspirated with low negative suction pressure (<100 cm H2O). Pooled BAL fluid was spun and the acellular supernatant frozen at −80° C.

DPPC was measured in BALF as described elsewhere (Heiring et al., 2020). In brief, the analysis of thawed acellular BALF (130 ul) was performed by dry Fourier Transform Infrared (FTIR) Spectroscopy. Spectra were obtained by a Simedx Alpha+ (London, UK) device equipped with a Perkin Elmer SP-2 spectrometer (20 scans; resolution: 4 cm-1; aperture: 10 mm). Lamellar bodies were spun down at 4000×g and the precipitate was transferred to a CaF2 disk, dried, and a spectrum was obtained from which the concentration of DPPC was derived.

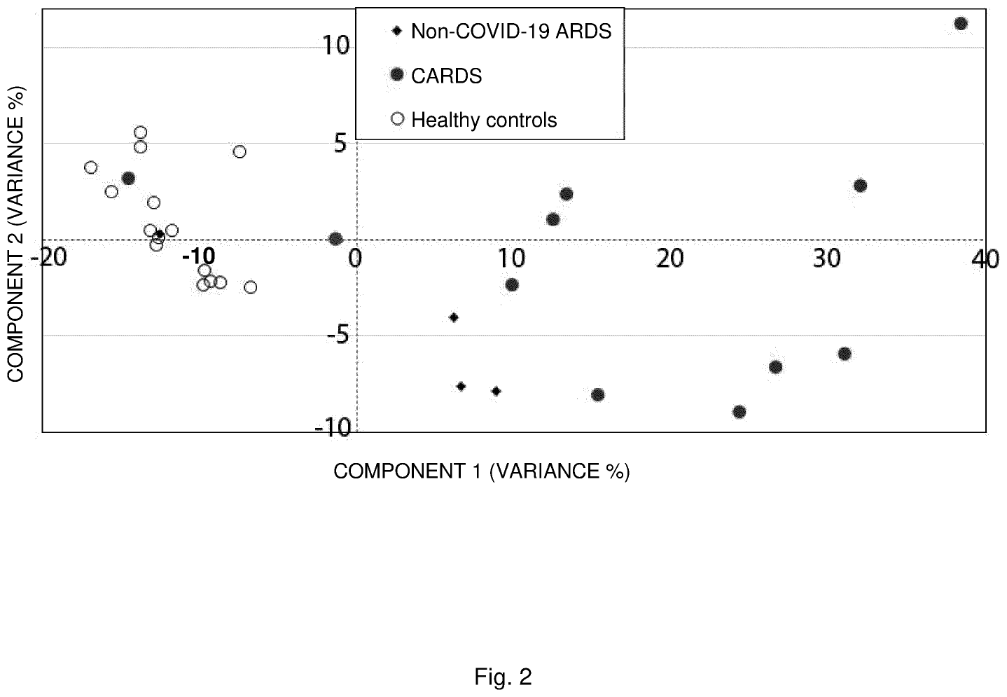

The mathematical algorithm used to obtain predicted DPPC levels from spectra was constructed from surfactant reference samples previously assessed by mass spectrometry (Heiring et al., 2020) and spectral data were analysed by principal component analysis (PCA) (FIG. 2). DPPC values between the three groups were compared using non-parametric Kruskal Wallis test, whereas a Mann-Whitney U-test was used to compared two groups. Algorithm development and statistical analysis was performed in R software, version 4.0.3/4.05.

Results

All patients had moderate-to-severe impairment of oxygenation at the time of BAL procedure. The overall mortality was high, both in CARDS (6/11) and non-COVID-19 ARDS (2/4).

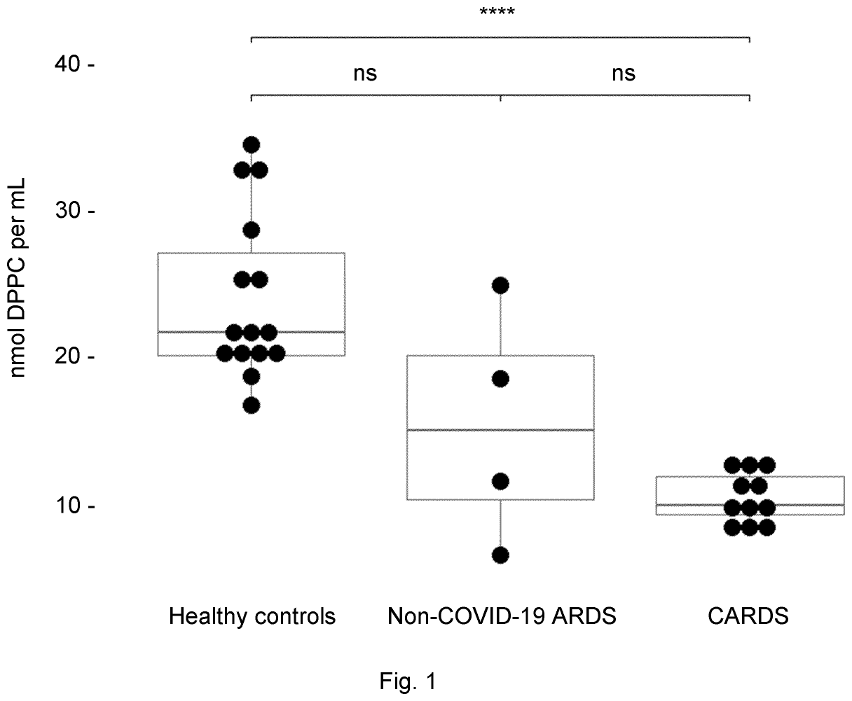

DPPC values were different across the three groups (FIG. 1, P<0.0001) with approximately 60% lower levels in CARDS than in HCs. In non-COVID-19 ARDS, DPPC tended to be lower than in HCs (P=0.051) but did not differ from CARDS (P=0.327). Within group analysis of DPPC levels did not show a difference between dexamethasone naïve (n=4) and dexamethasone treated (n=7) CARDS (P=0.30, data not shown).

A PCA model based on normalized baseline-corrected spectral data is shown in FIG. 2. The PCs separated the data into several clusters with a clear, visual separation of HCs and CARDS. HC spectra in general were much less scattered due to a low variability compared to CARDS.

It is expected that a similar relationship between DPPC values in the three analyzed groups will be observed if DPPC is instead measured in tracheal secretion samples instead of bronchoalveolar lavage samples.

The data suggest that pulmonary surfactant is critically suppressed in COVID-19 ARDS patients, and which may severely impose alveolar collapse, impair gas exchange, and increase the work of breathing. These changes could also predispose to barotrauma e.g. pneumothorax, which has increasingly been reported in larger cohorts of CARDS. These patients thus have potential for clinical benefit of exogenous surfactant administration.

1. A method for predicting the likelihood of acute respiratory distress syndrome (ARDS) in an adult subject, the method comprising the steps of:
a) providing a sample obtained from said subject,
b) determining in the sample of a) using suitable analysis means, the amount of at least a first and optionally at least a second compound, wherein the first compound is different than the second compound;
c) obtaining a concentration of the first compound and/or a ratio between the first and the second compounds of b); and
d) correlating the concentration of c) with a control concentration, wherein a concentration lower than or equal to the control concentration is indicative of ARDS of the subject; and/or correlating the ratio of c) with a control ratio, wherein a ratio equal to or lower than the control ratio is indicative of ARDS of the subject;
wherein the first compound is lecithin or saturated lecithin, and the second compound is sphingomyelin.

2. The method according to claim 1, comprising the steps of:
a) providing a sample obtained from said subject,
b) determining in the sample of a), the amount of at least a first and at least a second compound, wherein the first compound is different than the second compound;
c) obtaining a ratio between the first and the second compounds of b); and
d) correlating the ratio of c) with a control ratio, wherein a ratio equal to or lower than the control ratio is indicative of ARDS of the subject;
wherein the first compound is lecithin or saturated lecithin, and the second compound is sphingomyelin.

3. The method of any one of the preceding claims, wherein the first compound is dipalmitoylphosphatidylcholine (DPPC).

4. The method according to any one of the preceding claims, wherein the subject is suffering from a disease or a condition that can cause ARDS.

5. The method according to claim 4, wherein said disease or condition is selected from the group consisting of pneumonia, such as viral or bacterial pneumonia, acute inhalation injury, radiation-induced lung injury, aspiration, pulmonary embolism, atelectasis, emphysema, cystic fibrosis, chronic obstructive pulmonary disease, lung contusion, chest trauma, near-drowning, sepsis, shock, trauma, cardiopulmonary bypass, transfusion-related acute lung injury, burns, and increased intracranial pressure.

6. The method according to any one of the preceding claims, wherein said disease or condition is pneumonia.

7. The method according to any one of the preceding claims, wherein said disease is caused by an influenza virus or a coronavirus, such as wherein the disease is caused by a betacoronavirus such as SARS, MERS, or COVID-19.

8. The method according to any one of the preceding claims, wherein the sample is a lung epithelial lining fluid sample, a tracheal secretion sample, an oropharyngeal secretion sample, a gastric aspirate sample, a bronchoalveolar lavage sample, a pleural fluid sample, or a blood sample.

9. The method according to any one of the preceding claims, wherein the sample is a bronchoalveolar lavage sample.

10. The method according to claim 9, wherein the bronchoalveolar lavage sample is obtained less than 72 hours after intubation of the subject, such as less than 48 hours after intubation of the subject, such as less than 24 hours after intubation of the subject, such as less than 12 hours after intubation of the subject.

11. The method according to any one of the preceding claims, wherein the sample is directly transferred from the subject by a sampling means to an analysis means, without intermediate sample preparation, or further comprising a step of filtering, centrifuging, sonicating, adding an additive such as tris(2-carboxyethyl)phosphine (TCEP) to the sample, and/or diluting the sample prior to loading the sample onto the analysis means.

12. The method according to any one of the preceding claims, wherein step b) comprises the steps of:
i. homogenising and diluting said sample in a first volume of a first solution, thereby obtaining a homogenous sample;
ii. centrifuging the sample of step i) to obtain a pellet comprising lamellar bodies and a supernatant;
iii. optionally discarding the supernatant and optionally resuspending the pellet in a second volume of a second solution, thereby obtaining a sample for analysis; and
iv. determining the amount of the first compound, and the amount of the second compound, using analysis means.

13. The method according to any one of the preceding claims, wherein the sample has a volume between 10 and 1000 μL, such as between 30 and 900 μL, such as between 60 and 800 μL, such as between 80 and 600 μL, such as between 100 and 500 μL, such as between 150 and 450 μL, such as between 200 and 400 μL, such as between 250 and 350 μL, such as 250 μL, 300 μL or 350 μL.

14. The method according to any of the preceding claims, wherein the sample provided in step a) is homogenous.

15. The method according to any one of claims 12 to 14, wherein the first solution is a hypotonic solution or saline solution.

16. The method according to claim 15, wherein the first solution is a hypotonic solution such as water or deionised water.

17. The method according to claim 15, wherein the first solution is saline solution.

18. The method according to any one of claims 12 to 17, wherein the ratio of the volume of the sample of step a) to the volume of the first solution used in step i) is between 1:1 and 1:10, such as 1:1, 1:2, 1:3, 1:4, 1:5, 1:6, 1:7, 1:8, 1:9 or 1:10, preferably 1:4 or 1:6.

19. The method according to any one of claims 12 to 18, wherein an additive such as tris-(2-carboxyethyl)fosfin, (TCEP) is added to the sample prior to homogenising in step i).

20. The method according to any one of claims 12 to 19, wherein homogenising in step i) is performed by pipetting repeatedly or vortexing.

21. The method according to any one of claims 12 to 20, wherein step i) further comprises centrifuging the sample after homogenisation and discarding the supernatant.

22. The method according to claim 21, wherein the centrifugation after homogenisation in step i) is performed at a force between 100 g and 500 g, such as 300 g.

23. The method according to any one of claims 12 to 22, wherein the centrifugation of step ii) is performed at a force between 500 g and 10000 g, such as 4000 g.

24. The method according to any one of the claims 12 to 23, wherein the centrifugation of step ii) is performed for a duration of 1 min to 10 min, such as 2 min to 9 min, such as 3 min to 8 min, such as 4 min to 7 min, such as 5 min to 6 min, such as 4 min, 5 min or 6 min.

25. The method according to any of the preceding claims 12 to 24, wherein the centrifugation of step ii) is performed at 4000 g for 4 min.

26. The method according to any one of claims 12 to 25, wherein discarding the supernatant in step i) and/or step iii) is performed by pipetting the supernatant or by pouring away the supernatant.

27. The method according to any one of claims 12 to 26, wherein resuspending the pellet in step iii) is performed by pipetting repeatedly or vortexing.

28. The method according to any one of claims 12 to 27, wherein the second solution is a hypotonic solution.

29. The method according to claim 28, wherein the second solution is a hypotonic solution such as water or deionised water.

30. The method according to any one of claims 12 to 29, wherein the second volume is between 10 and 200 μL, such as between 25 and 175 μL, such as between 50 and 150 μL, such as between 75 and 125 μL, such as 100 μL, 75 μL, 50 μL, or 25 μL.

31. The method according to any one of claims 12 to 30, wherein step iii) further comprises a step of drying the sample after resuspension, whereby the second solution is at least partially removed by evaporation.

32. The method according to any one of claims 12 to 31, wherein step iv) further comprises a step of drying the sample prior to determining the amount of the first compound.

33. The method according to any one of claims 12 to 32, wherein step iv) further comprises a step of transferring the sample for analysis to a support structure such as a CaF₂ window, optionally wherein the support structure is at a temperature allowing for at least partial evaporation of the second solution, such as 90° C.

34. The method according to any one of the preceding claims, wherein step b) comprises the steps of:
i. adding an additive such as tris-(2-carboxyethyl)fosfin, (TCEP) to the sample, homogenising and centrifuging the sample, discarding the supernatant and diluting said sample in a first volume of a first solution, thereby obtaining a homogenous sample;
ii. centrifuging the sample of step i) to obtain a pellet comprising lamellar bodies and a supernatant;
iii. discarding the supernatant and drying the sample; and
iv. determining the amount of the first compound, and the amount of the second compound, using analysis means.

35. The method according to any one of the preceding claims, wherein the analysis means is an infrared spectrometer, such as a Fourier transformed infrared (FTIR) spectrometer.

36. The method according to any one of the preceding claims wherein the analysis means is an FTIR spectrometer.

37. The method according to any one of the preceding claims, wherein the analysis is performed by Fourier transformed infrared spectroscopy (FTIR) and/or attenuated total reflectance (ATR).

38. The method according to any one of the preceding claims, wherein the amount of the first compound and optionally the amount of the second compound are determined in the mid-wavelength infrared range.

39. The method according to any one of the preceding claims, further comprising determining the amount of a third compound.

40. The method according to claim 39, wherein said third compound is selected from the group consisting of phosphatidylglycerol, hemoglobin, apo-hemoglobin, heme and porphyrin, preferably the third compound is phosphatidylglycerol or hemoglobin.

41. The method according to any one of the preceding claims, wherein the amount of the first, second and/or third compound is determined by measuring the activity and/or concentration of the compound(s).

42. The method according to any one of claims 39 to 41, further comprising the step of subtracting the amount of the third compound from the amount of the first compound, thus obtaining a background corrected amount of the first compound.

43. The method according to any one of claims 39 to 42, further comprising the step of subtracting the amount of the third compound from the amount of the second compound, thus obtaining a background corrected amount of the second compound.

44. The method according to any one of claims 39 to 43, further comprising the step of subtracting the amount of the third compound from the amount of both the first and the second compound, thus obtaining a background corrected amount of both the first and the second compound.

45. The method according to any one of the preceding claims, wherein the method is temperature-independent, at least in the range of 20° C. to 40° C.

46. The method according to any one of the preceding claims, wherein the subject is a human being.

47. The method according to any one of the preceding claims, wherein the time-to-result of the method is between 5 and 60 minutes, such as between 8 and 30 minutes, such as 15 minutes or less.

48. The method according to any one of the preceding claims, wherein the time-to-result of the method is less than 10 minutes, such as about 5 minutes.

49. The method according to any one of the preceding claims, wherein the method allows for initiation of treatment within 5 to 60 minutes, such as within 10 to 50 minutes, such as within 20 to 40 minutes, such as within 25 to 35 minutes, such as within about 30 minutes from providing a sample in step a).

50. The method according to any one of the preceding claims, wherein the method allows for initiation of treatment within 60 minutes or less, such as within 55 minutes or less, such as within 50 minutes or less, such as within 45 minutes or less, such as within 40 minutes or less, such as within 35 minutes or less, such as within 30 minutes or less, such as within 25 minutes or less, such as within 20 minutes or less, such as within 15 minute or less, such as within 10 minutes or less, such as within 5 minutes or less from providing a sample in step a).

51. The method according to any one of the preceding claims, wherein the control ratio is between 1.0 and 10.0±0.5, such as between 1.5 and 9.0±0.5, such as between 2.0 and 8.0±0.5, such as between 2.0 and 7.0±0.5, such as between 2.0 and 6.0±0.5, such as 2.5±0.5.

52. The method according to any one of the preceding claims, wherein the control ratio is between 2.0 and 5.0±0.5, such as between 2.0 and 4.5±0.5, such as between 2.0 and 4.0±0.5, such as between 2.0 and 3.5±0.5, such as between 2.0 and 3.2±0.5, such as 3.0±0.5.

53. The method according to any one of the preceding claims, wherein if the ratio between the first compound and the second compound is equal to or less than 10.0±0.5, such as equal to or less than 9.0±0.5, such as equal to or less than 8.0±0.5, such as equal to or less than 7.0±0.5, such as equal to or less than 6.0±0.5, such as equal to or less than 5.0±0.5, such as equal to or less than 4.0±0.5, such as equal to or less than 3.0±0.5, such as equal to or less than 2.0±0.5, or such as equal to or less than 1.0±0.5, the subject is classified as having or likely to develop ARDS.

54. The method according to any one of the preceding claims, wherein if the concentration of the first compound is equal to or less than 25 μM±0.5 μM, equal to or less than 20 μM±0.5 μM, equal to or less than 17.5±0.5 μM, equal to or less than 15±0.5 μM, or equal to or less than 12.5±0.5 μM, the subject is classified as having or likely to develop ARDS.

55. The method according to any one of the preceding claims, wherein the method has a specificity of 50% or more, such as 60% or more, such as 70% or more, such as 80% or more, such as 90% or more.

56. The method according to any one of the preceding claims, wherein the method has a sensitivity of 50% or more, such as 60% or more, such as 70% or more, such as 80% or more, such as 90% or more.

57. A method of monitoring the progression of disease in a subject, said method comprising performing the method according to any one of the preceding claims, wherein said sample is provided from the subject every 72 hours or less, such as every 60 hours or less, such as every 48 hours or less, such as every 36 hours or less, such as every 24 hours or less, such as every 20 hours or less, such as every 16 hours or less, such as every 12 hours or less, such as every 8 hours or less, such as every 6 hours or less, such as every 4 hours or less, such as every 2 hours or less, such as every 1 hour or less.

58. The method according to any one of the preceding claims, wherein said method is used to determine the likelihood of said subject requiring ventilatory support.

59. A method for predicting the likelihood of an adult subject benefitting from administration of surfactant, said method comprising performing the method as defined in any one of claims 1 to 57,
wherein a concentration determined in step c) lower than or equal to the control concentration is indicative of the subject benefitting from administration of surfactant; and/or wherein a ratio determined in step c) equal to or lower than the control ratio is indicative of the subject benefitting from administration of surfactant.

60. The method according to claim 59, wherein a concentration equal to or less than 25 μM±0.5 μM, equal to or less than 20 μM±0.5 μM, equal to or less than 17.5±0.5 μM, equal to or less than 15±0.5 μM, or equal to or less than 12.5±0.5 μM, indicates that the subject is likely to benefit from administration of surfactant.

61. A computer implemented method for predicting the likelihood of ARDS in an adult subject based on spectral data acquired from a sample obtained from said subject, the method comprising the steps of:
a) determining the activity and/or concentration of a first and optionally a second compound in said sample by analysing said spectral data, wherein the first compound is lecithin or saturated lecithin and the second compound is sphingomyelin;
b) optionally calculating a ratio between the activities and/or concentrations of the first and the second compound;
c) correlating said concentration with a control value and/or correlating said ratio with a control ratio, wherein a concentration lower than or equal to the control concentration is indicative of ARDS of the subject and/or a ratio equal to or lower than the control ratio is indicative of ARDS of the subject.

62. The computer implemented method of claim 61 comprising the steps of:
a) determining the activity and/or concentration of a first and a second compounds in said sample by analysing said spectral data, wherein the first compound is lecithin or saturated lecithin and the second compound is sphingomyelin;
b) calculating a ratio between the activities and/or concentrations of the first and the second compound;
c) correlating said ratio with a control ratio, wherein a ratio equal to or lower than the control ratio is indicative of ARDS of the subject.

63. The method according to any one of claims 61 to 62, further comprising the features of any of claims 1 to 57.

64. A computer program product having a computer readable medium, said computer program product suitable for predicting the likelihood of ARDS in an adult subject based on spectral data acquired from a sample obtained from said subject, said computer program product comprising means for carrying out all the steps of the method as defined in one of claims 61 to 63.

65. A system for predicting the likelihood of ARDS in an adult subject based on a sample obtained from said subject, comprising:
a spectroscope for measuring spectral data from said sample, and
processing means configured for:
a) determining the activity and/or concentration of a first and optionally a second compound in said sample by analysing said spectral data, wherein the first compound is lecithin or saturated lecithin and the second compound is sphingomyelin;
b) optionally calculating a ratio between the activities and/or concentrations of the first and the second compound;
c) correlating said concentration with a control value and/or correlating said ratio with a control ratio, wherein a concentration lower than or equal to the control concentration is indicative of ARDS of the subject and/or a ratio equal to or lower than the control ratio is indicative of ARDS of the subject; and
d) indicating whether the ratio is equal to or lower than the control ratio, wherein a ratio equal to or lower than a predefined value is indicative of ARDS of the subject.

66. The system according to claim 65, wherein the processing means are configured for:
a) determining the activity and/or concentration of a first and a second compounds in said sample by analysing said spectral data, wherein the first compound is lecithin or saturated lecithin and the second compound is sphingomyelin;
b) calculating a ratio between the activities and/or concentrations of the first and the second compound;
c) correlating said ratio with a control ratio, wherein a ratio equal to or lower than the control ratio is indicative of ARDS of the subject; and
d) indicating whether the ratio is equal to or lower than the control ratio, wherein a ratio equal to or lower than a predefined value is indicative of ARDS of the subject.

67. Use of a system in a method for predicting the likelihood of ARDS in an adult subject according to any one of claims 1 to 57, said system comprising:
a spectroscope for measuring spectral data from said sample, and
processing means configured for:
a) determining the activity and/or concentration of a first and optionally a second compound in said sample by analysing said spectral data, wherein the first compound is lecithin or saturated lecithin and the second compound is sphingomyelin;
b) optionally calculating a ratio between the activities and/or concentrations of the first and the second compound;
c) correlating said concentration with a control value and/or correlating said ratio with a control ratio, wherein a concentration lower than or equal to the control concentration is indicative of ARDS of the subject and/or a ratio equal to or lower than the control ratio is indicative of ARDS of the subject; and
d) indicating whether the ratio is equal to or lower than the control ratio, wherein a ratio equal to or lower than a predefined value is indicative of ARDS of the subject.

68. Use of a system according to claim 67, said system comprising:
a spectroscope for measuring spectral data from said sample, and
processing means configured for:
a) determining the activity and/or concentration of a first and a second compounds in said sample by analysing said spectral data, wherein the first compound is different than the second compound and the first compound is lecithin or saturated lecithin and the second compound is sphingomyelin;
b) calculating a ratio between the activities and/or concentrations of the first and the second compounds;
c) correlating said ratio with a control ratio; and
d) indicating whether the ratio is equal to or lower than the control ratio, wherein a ratio equal to or lower than a predefined value is indicative of ARDS of the subject.

69. The method according to any one of claims 1 to 60 further comprising administering a treatment to a subject having an increased likelihood of ARDS.

70. The method according to claim 69, wherein the treatment comprises continuous positive airway pressure (CPAP), mechanical ventilation, such as airway pressure release ventilation or positive end-expiratory pressure (PEEP) ventilation, extracorporeal membrane oxygenation, fluid management, such as fluid restriction or inducing diuresis, administering surfactant, administering a corticosteroid, administering nitric oxide, administrating supplemental oxygen and/or administering an antiviral compound, such as remdesivir.

71. The method according to claim 69, wherein the treatment comprises administering surfactant or one or more components thereof, such as dipalmitoylphosphatidylcholine (DPPC), phosphatidylcholine, phosphatidylglycerol, cholesterol, surfactant protein A, surfactant protein B, surfactant protein C and/or surfactant protein D, to the subject, such as by nebulization or bolus.

72. The method according to claim 71, wherein said surfactant is a synthetic surfactant, an animal-derived surfactant, or a combination of a synthetic surfactant and an animal-derived surfactant.

73. Surfactant for use in the treatment of ARDS in a subject, wherein the surfactant is administered to a subject having an increased likelihood of ARDS as determined by any of claims 1 to 57.

74. Use of a surfactant for the manufacture of a medicament for the treatment of ARDS in a subject, wherein the surfactant is administered to a subject having an increased likelihood of ARDS as determined by any of claims 1 to 57.
